(19) United States
(12) Patent Application Publication
(10) Pub. No.: US 2023/0210722 A1
(43) Pub. Date: Jul. 6, 2023

(54) EPINEPHRINE PREMIX FORMULATIONS AND USES THEREOF

(71) Applicants: Baxter International, Inc., Deerfield, IL (US); Baxter Healthcare SA, Glattpark (Opfikon) (CH)

(72) Inventors: Dipakkumar Thakordas Thakker, Panchmahal (IN); Pankajkumar Ramabhai Patel, Ahmedabad (IN); Vijaykumar Amrutlal Patel, Ahmedabad (IN); Riyazkhan Moula Mulani, Ahmedabad (IN)

(21) Appl. No.: 18/145,813

(22) Filed: Dec. 22, 2022

(30) Foreign Application Priority Data
Dec. 29, 2021 (IN) 202141061522

(51) Int. Cl. A61J 1/14; A61K 31/137; A61K 9/00; A61K 47/18; A61K 47/02

(52) U.S. Cl. CPC A61J 1/1468; A61K 31/137; A61K 9/0019; A61K 47/183; A61K 47/02

(57) ABSTRACT

A pharmaceutical premix formulation comprising from about 10 mcg/ml to about 70 mcg/ml epinephrine and a salt is described, wherein the formulation is an aqueous, premix formulation and has a pH from about 2 to about 6. Uses and systems including the epinephrine premix formulation are also provided.

### RELATED APPLICATIONS

This application claims priority to Indian Application No. 202141061522, filed on Dec. 29, 2021. The contents of the foregoing application are hereby incorporated by reference herein,

### TECHNICAL FIELD

The present disclosure generally relates to stable liquid epinephrine pharmaceutical premix formulations. The present disclosure also relates to methods of making and using liquid epinephrine pharmaceutical premix formulations, including in treating septic shock associated hypotension or anaphylaxis in a human subject in need thereof.

### BACKGROUND

Epinephrine is an endogenous adrenergic neurotransmitter synthesized and stored in the adrenal medulla and acts directly on both alpha- and beta-adrenergic receptors. Epinephrine is one of the neural hormones responsible for the regulation of the heart, blood pressure, airway resistance, and energy metabolism. It is classified as a sympathomimetic drug. Epinephrine generates an inotropic effect, wherein it increases the heart rate and the force of contraction of the heart, narrows the blood vessels thus increasing blood pressure, reduces airway resistance to make it easier to breathe, and raises blood glucose and blood fatty acids to supply energy to the body during stress.

Epinephrine can be used in emergency treatment of allergic reactions (Type I), including anaphylaxis, which may result from insect stings or bites, foods, drugs, sera, diagnostic testing substances and other allergens, as well as idiopathic anaphylaxis or exercise-induced anaphylaxis. Epinephrine is indicated to increase mean arterial blood pressure in adult patients with hypotension associated with septic shock.

Epinephrine injection is generally available as a concentrated solution and is administered as a bolus dose through a subcutaneous, intramuscular route. For intravenous delivery, a concentrated solution of epinephrine needs to be diluted (e.g., with 5% dextrose) before administration as a continuous intravenous infusion to achieve the desired concentration. For continuous intravenous administration, a medical professional has to prepare a diluted product solution with 5% dextrose injection or 5% dextrose and sodium chloride solution. Once the diluted solution is prepared, it typically is not held for more than 4 hours at room temperature or for 24 hours under refrigerated conditions. Thus, given the often urgent conditions under which epinephrine is being delivered to a patient, improvements in delivery are needed.

### SUMMARY

Typically, epinephrine must be diluted prior to administration to a human patient. The requisite dilution is inconvenient, time consuming, and can be subject to errors and product contamination. Therefore, a need exists for an improved epinephrine formulation that is stable, has an appropriate pH and osmolality, and contains an appropriate amount of epinephrine. Described herein are epinephrine formulations that are premixed and ready for infusion to the patient.

Disclosed herein is a pharmaceutical premix formulation comprising about 10 micrograms per milliliter (mcg/ml) to about 70 mcg/ml epinephrine, and a salt, wherein the formulation is an aqueous, premix formulation and has a pH from about 2 to about 6.

Disclosed herein is a pharmaceutical premix formulation comprising about 10 micrograms per milliliter (mcg/ml) to about 70 mcg/ml epinephrine, a salt, edetate disodium dihydrate (EDTA), and sodium metabisulfite, wherein the formulation is an aqueous, premix formulation and has a pH from about 2 to about 6.

In one embodiment, the pharmaceutical premix formulation comprises from about 16 mcg/ml to about 64 mcg/ml epinephrine. In one embodiment, the pharmaceutical premix formulation comprises about 16 mcg/ml epinephrine. In another embodiment, the pharmaceutical premix formulation comprises about 32 mcg/ml epinephrine. In one embodiment, the pharmaceutical premix formulation comprises about 64 mcg/ml epinephrine.

In one embodiment, the salt is sodium chloride. In one embodiment, the pharmaceutical premix formulation comprises from about 8.5 mg/ml to about 9.5 mg/ml sodium chloride. In one embodiment, the pharmaceutical premix formulation comprises from about 8.7 mg/ml to about 9.3 mg/ml. In one embodiment, the pharmaceutical premix formulation comprises about 9 mg/ml sodium chloride. In one embodiment, the pharmaceutical premix formulation comprises about 0.9% sodium chloride by weight per volume (w/v).

In one embodiment, the pharmaceutical premix formulation has a pH from about 2.2 to about 5.5.

In one embodiment, the pharmaceutical premix formulation further comprises edetate disodium dihydrate. In one embodiment, the pharmaceutical premix formulation comprises from about 0.01 to about 0.3 mg/ml edetate disodium dihydrate. In one embodiment, the pharmaceutical premix formulation comprises about 0.032 mg/ml edetate disodium dihydrate.

In one embodiment, the pharmaceutical premix formulation further comprises sodium metabisulfite. In one embodiment, the pharmaceutical premix formulation comprises from about 0.03 to about 0.07 mg/ml sodium metabisulfite. In one embodiment, the pharmaceutical premix formulation comprises about 0.05 mg/ml sodium metabisulfite. In one embodiment, the pharmaceutical premix formulation comprises a molar ratio of epinephrine to sodium metabisulfite, measured as sulfite-equivalents, in the range of 0.17 to 0.66.

Also provided herein is a pharmaceutical premix formulation consisting essentially of epinephrine, sodium chloride, edetate disodium dihydrate, and sodium metabisulfite, wherein the pharmaceutical premix formulation is an aqueous, premix formulation and has a pH from about 2.2 to about 5.5. In certain embodiments, the formulation comprises a molar ratio of epinephrine to sodium metabisulfite, measured as sulfite-equivalents, in the range of 0.17 to 0.66. In one embodiment, the pharmaceutical premix formulation comprises from about 10 mcg/ml to about 70 mcg/ml epinephrine. In one embodiment, the pharmaceutical premix formulation comprises from about 16 mcg/ml to about 64 mcg/ml epinephrine. In one embodiment, the pharmaceutical premix formulation comprises from about 8.4 mg/ml to about 9.4 mg/ml sodium chloride. In one embodiment, the pharmaceutical premix formulation comprises from about 0.01 to about 0.3 mg/ml edetate disodium dihydrate and/or from about 0.03 to about 0.07 mg/ml sodium metabisulfite.

Further described herein is a pharmaceutical premix formulation consisting essentially of about 16 mcg/ml epinephrine, about 0.9% sodium chloride, about 0.032 mg/ml edetate disodium dihydrate, and about 0.05 mg/ml sodium metabisulfite, wherein the pharmaceutical premix formulation is an aqueous, premix formulation and has a pH from about 2.2 to about 5.5.

The current disclosure also provides a pharmaceutical premix formulation consisting essentially of about 32 mcg/ml epinephrine, about 0.9% sodium chloride, about 0.032 mg/ml edetate disodium dihydrate, and about 0.05 mg/ml sodium metabisulfite, wherein the pharmaceutical premix formulation is an aqueous, premix formulation and has a pH from about 2.2 to about 5.5.

Also disclosed herein is a pharmaceutical premix formulation consisting essentially of about 64 mcg/ml epinephrine, about 0.9% sodium chloride, about 0.032 mg/ml edetate disodium dihydrate, and about 0.05 mg/ml sodium metabisulfite, wherein the pharmaceutical premix formulation is an aqueous, premix formulation and has a pH from about 2.2 to about 5.5.

An advantage of the pharmaceutical premix formulations described herein is that they are stable. In one embodiment, the pharmaceutical premix formulation described herein is stable at about 25 degrees Celsius for at least about 6 months. In one embodiment, the pharmaceutical premix formulation described herein is stable at about 25 degrees Celsius for about 12 months. In one embodiment, the pharmaceutical premix formulation described herein is stable at about 21-22 degrees Celsius for more than about 4 hours. In one embodiment, the pharmaceutical premix formulation described herein is stable at about 25 degrees Celsius for about 24 hours or more at about 4 degrees Celsius.

Also included herein are the pharmaceutical premix formulations described in the Examples and the Tables therein.

Further included are pharmaceutical premix formulations comprising epinephrine having low levels of impurities, including those described in Tables 1 to 23 (both amounts and types of impurities).

A pharmaceutical premix formulation described herein can be contained in a flexible container. In one embodiment, the flexible container is polypropylene (PP), polyamide (PA), or polyethylene (PE). In one embodiment, the flexible container has a volume of about 100 mL or about 250 mL.

Also provided herein is a system comprising an oxygen scavenger and a pharmaceutical premix formulation described herein. In one embodiment, the oxygen scavenger is in a polyethylene container. In one embodiment, the oxygen scavenger comprises micronized iron.

Further provided herein is a method of treating hypotension in a human subject in need thereof, the method comprising intravenously administering a pharmaceutical premix formulation disclosed herein to the human subject in need thereof, wherein the pharmaceutical premix formulation is not diluted prior to intravenous administration to the human subject. In one embodiment, the human subject has anaphylaxis or hypotension associated with septic shock. In one embodiment, the formulation is administered as an intravenous bolus. In one embodiment, the formulation is administered as a continuous intravenous infusion.

### DETAILED DESCRIPTION

A goal of the present invention is to provide a ready to use epinephrine injectable (infusion) solution in order to minimize medication errors, improve formulation stability, and to make administration of the epinephrine solution easier for medical professionals for use in an infusion regimen. This is important given the types of indications for which intravenous (IV) epinephrine is used, such as, but not limited to, anaphylaxis and hypotension associated with septic shock.

Disclosed herein are room temperature stable, ready to use premix formulations of epinephrine in sodium chloride. The premix formulations may be contained within a container closure system, e.g., 250 mL VIAFLO container closure system. The premix formulations described herein are already diluted forms of epinephrine, e.g., about 10 μg/ml to about 70 μg/ml (about 10 mcg/ml to about 70 mcg/ml), which can be used for an intravenous bolus or as a continuous infusion depending on the dosage requirement.

The present disclosure is generally directed to a pharmaceutical premix formulation comprising from about 10 mcg/ml to about 70 mcg/ml of epinephrine, wherein the formulation is an aqueous, premix formulation and has a pH of about 2 to about 6. A liquid epinephrine premix pharmaceutical formulation, as described herein, is ready for administration without the need for dilution to achieve a certain concentration suitable for administration to a human patient. The formulation of the disclosure can be administered to a human patient in need thereof intravenously, e.g., as either a bolus intravenous dose or by continuous intravenous infusion. The liquid epinephrine premix pharmaceutical formulation described herein may be aseptically filled into a container or terminally sterilized. The container is preferably a flexible container and forms a sterile pharmaceutical epinephrine premix product when filled with the premix formulation. The liquid epinephrine premix pharmaceutical formulation can be a single use premix which is a sterile, stable, and ready to use aqueous solution for intravenous (IV) administration, such as IV infusion, including continuous infusion.

The epinephrine formulations described herein are ready to use injectable solutions. The disclosed premix pharmaceutical epinephrine formulation and containers containing said formulation are advantageous in reducing the risk of contamination and errors associated with dilution, providing time savings, convenience, and reducing waste by eliminating the need for such dilution. The epinephrine formulations provided herein are ready to use premix formulations to minimize potential for any medication errors and to make it easier for medical professionals to administer epinephrine formulations by infusion regimens.

### Definitions

With respect to the terms used in this disclosure, the following definitions are provided. This application will use the following terms as defined below unless the context of the text in which the term appears requires a different meaning.

As used herein, the term “premix” refers to a ready to use, liquid solution suitable for direct administration to human patients, including by intravenous (IV) infusion, without requiring dilution of the formulation prior to administration. “Premix” indicates the formulation is already mixed and is suitable for administration to a human patient. Preferably, the premix solution is supplied as a sterile solution, and is stable over its shelf life, as described herein. “Liquid solution” indicates the formulation is a homogenous, liquid phase mixture, and explicitly excludes other formats, such as solid or gel.

The term “pharmaceutical formulation” refers to a preparation which is in such form as to permit the biological activity of an active ingredient, e.g., epinephrine, contained therein to be effective, and which contains no additional components which are unacceptably toxic to a subject to which the formulation would be administered.

The term “aqueous” when used in reference to a formulation refers to a liquid formulation in which the solvent is water (e.g., water for injection (WFI)).

The term “pharmaceutically acceptable” as used herein refers to substances that do not cause substantial adverse allergic or immunological reactions when administered to a human subject.

As used herein, the term “sterile” is understood to mean free from any bacteria or other living microorganisms.

A “stable” formulation is one in which the active ingredient therein, e.g., epinephrine, essentially retains its physical and chemical stability, therefore its biological activity, upon storage.

A “patient”, “subject” or “individual”, used interchangeably herein, is a mammal, preferably a human.

As used herein, an “effective amount” is an amount effective, at dosages, and for periods of time necessary, to achieve the desired result with respect to the treatment of the relevant disorder, condition, or side effect. In one embodiment, an effective amount of epinephrine is an amount that provides relief from a patient from anaphylaxis or hypotension associated with septic shock.

The terms “substantially no,” “essentially free” or “substantially free” as used in reference to a particular component means that any of the component present constitutes less than about 3.0% by weight, such as less than about 2.0% by weight, less than about 1.0% by weight, preferably less than about 0.5% by weight or, more preferably, less than about 0.1% by weight.

The phrase “consists essentially of” as used herein in reference to a premix formulation, means that the formulation necessarily includes the listed ingredients (including the active ingredient), and is open to unlisted ingredients that do not materially affect the basic nature or stability of the premix formulation, e.g., liquid, sterile, or activity of the active ingredient, e.g., epinephrine.

### Epinephrine Premix Pharmaceutical Formulations and Uses Thereof

A pharmaceutical premix formulation disclosed herein provides multiple advantages over the art. Epinephrine is mainly administered by intramuscular, subcutaneous, and intravenous modes. Generally, epinephrine injection is available as a concentrated solution which needs to be diluted before administration to a human patient as an intravenous bolus or continuous intravenous infusion to achieve the desired concentration. The concentrated epinephrine formulation, for example, generally needs to be diluted with 0.9% sodium chloride or 5% dextrose in water. Once the diluted solutions are prepared, they are typically not held for more than 4 hours at room temperature or 24 hours under refrigerated conditions.

Disclosed herein are premix, liquid formulations having amounts of epinephrine that are suitable for administering to a human patient without first diluting the formulation. Generally, the pharmaceutical premix formulation described herein has a low concentration of epinephrine, e.g., less than about 70 μg/ml (e.g., 16 to 64 μg/ml) and does not require dilution prior to be administered to a patient. A pharmaceutical premix formulation described herein has an epinephrine concentration suitable to administer a desired effective dose to a human patient—thus improving efficiency and also minimizing potential for error that may result from the dilution process.

The pharmaceutical premix formulations described herein include epinephrine. Epinephrine is a sympathomimetic catecholamine, which may also be referred to as adrenaline. Chemically, epinephrine is ((−)-3,4-Dihydroxy-α-[(methylamino)methyl]benzyl alcohol), with a chemical formula of C₉H₁₃NO₃ and a structural formula as follows:

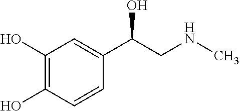

Epinephrine may be in the form of the free base, or may be in the form of a salt, such as a hydrochloride, sulfate, or bitartrate. Unless otherwise indicated, epinephrine refers to epinephrine bitartrate, and amounts of epinephrine provided throughout are on a free base epinephrine form (free base epinephrine equivalent).

Presented herein are premix, aqueous pharmaceutical formulations which are stable and contain epinephrine. Notably, the pharmaceutical premix formulations described herein do not need to be diluted prior to administration to a patient. Further, they are stable at room temperature. Preferably, the formulation is an aqueous formulation.

Described herein is a pharmaceutical premix formulation comprising from about 10 mcg/ml to about 70 mcg/ml epinephrine and a salt, wherein the formulation is an aqueous, premix formulation and has a pH from about 2 to about 6. In one embodiment, sodium chloride is used at a concentration of about 0.9% w/v.

In one embodiment, each mL of a pharmaceutical premix formulation is an aqueous formulation and comprises epinephrine bitartrate equivalent to epinephrine base: about 16 mcg/mL or about 32 mcg/mL or about 64 mcg/mL; sodium chloride: 9.0 mg/ml; edetate disodium dihydrate: about 0.2 mg/mL; sodium metabisulfite about 0.05 mg/mL; and sodium hydroxide and hydrochloric acid to adjust pH between about 2.2 and about 5.5, if necessary.

In a preferred embodiment, the salt used in the pharmaceutical premix formulation comprising epinephrine is sodium chloride. In one embodiment, the pharmaceutical premix formulation disclosed herein comprises epinephrine and about 8 to about 9.5 mg/ml sodium chloride. In other embodiments, the pharmaceutical premix formulation disclosed herein comprises epinephrine and about 8.5 to about 9.5 mg/ml sodium chloride. In other embodiments, the pharmaceutical premix formulation disclosed herein comprises epinephrine and about 8.7 to about 9.3 mg/ml sodium chloride. In one embodiment, the pharmaceutical premix formulation disclosed herein comprises epinephrine and about 9.0 mg/ml sodium chloride.

The amount of epinephrine or the amount of salt, e.g., sodium chloride, in the pharmaceutical premix formulation may be described in terms of percentage by weight per volume (w/v), e.g., about 0.9% sodium chloride, or by concentration in the liquid formulation, e.g., about 9.0 mg/ml sodium chloride. Concentrations of components of the formulation may also be expressed in terms of molarity (e.g., M or mM; the number of moles or millimoles per liter, respectively).

In one embodiment, the amount of salt, e.g., sodium chloride, is described in terms of the w/v% of the liquid solution. For example, the pharmaceutical premix formulation may contain about 0.7% to about 1.1% sodium chloride or about 0.9% sodium chloride w/v.

The osmolality of the pharmaceutical premix formulation comprising epinephrine should be suitable for administration, e.g., intravenous, to a human patient. For example, the formulation can have an osmolality from about 270 to about 330 mOsm/kg.

One feature of a pharmaceutical premix formulations described herein is the concentration of epinephrine, as the concentration is such that dilution of the epinephrine solution prior to administration is not required. The ready-to-use, stable liquid premix formulation described herein does not require mixing or diluting prior to delivery to a human patient. The ready-to-use, stable, liquid premix formulation has an epinephrine concentration ranging from about 10 mcg/ml to about 70 mcg/ml, such as about 10, about 15, about 16, about 17, about 18, about 19, about 20, about 25, about 30, about 31, about 32, about 33, about 34, about 35, about 40, about 45, about 50, about 55, about 60, about 61, about 62, about 63, about 64, about 65, or about 70 mcg/ml, more preferably from about 16 mcg/ml to about 64 mcg/ml, of epinephrine. Ranges including the amounts disclosed herein are also contemplated, e.g., about 15 to about 65 mcg/ml. In one embodiment, a pharmaceutical premix formulation described herein comprises about 16 mcg/ml epinephrine. In one embodiment, a pharmaceutical premix formulation described herein comprises about 32 mcg/ml epinephrine. In one embodiment, the concentration of epinephrine is about 64 mcg/ml.

In some embodiments, the concentration of epinephrine in the pharmaceutical premix formulations described herein may be expressed in terms of millimoles per liter (mM). Accordingly, in some embodiments, the ready-to-use, stable, liquid premix formulation has an epinephrine concentration ranging from about 0.055 mM to about 0.385 mM, corresponding to a range by weight per volume from about 10 mcg/ml to about 70 mcg/ml.

In some embodiments, the ready-to-use, stable, liquid premix formulation has an epinephrine concentration in a range from about 0.088 mM to about 0.352 mM, corresponding to a range by weight per volume from about 16 mcg/ml to about 64 mcg/ml. In some embodiment, the pharmaceutical premix formulation comprises epinephrine at a concentration of about 0.088 mM. In some embodiment, the pharmaceutical premix formulation comprises epinephrine at a concentration of about 0.176 mM. the pharmaceutical premix formulation comprises epinephrine at a concentration of about 0.352 mM.

As described in more detail below, a pharmaceutical premix formulation may be contained in a flexible bag, such as a non-polyvinyl chloride (NPVC) flexible container. In some embodiments, the ready to use formulation of epinephrine comprises a diluted amount of epinephrine (about 16 μg/ml, about 32 μg/ml or about 64 μg/ml (i.e., about 16 mcg/ml, about 32 mcg/ml, or about 64 mcg/ml) in about 0.9% sodium chloride injection in non-polyvinyl chloride (NPVC flexible container (e.g., 250 mL VIAFLO Container Closure System). In one embodiment, the pharmaceutical premix formulation comprises about 16 μg/ml epinephrine in about 0.9% sodium chloride injection in NPVC flexible container. In one embodiment, the pharmaceutical premix formulation comprises about 32 μg/ml epinephrine in about 0.9% sodium chloride injection in NPVC flexible container. In one embodiment, the pharmaceutical premix formulation comprises about 64 μg/ml epinephrine in about 0.9% sodium chloride injection in NPVC flexible container.

To provide hemodynamic support in septic shock associated hypotension in adult patients, the suggested dosing infusion rate of intravenously administered epinephrine is about 0.05 to about 2 mcg/kg/min and is titrated to achieve a desired mean arterial pressure (MAP). The dosage may be adjusted periodically, such as every 10-15 minutes, in increments of about 0.05 to about 0.2 mcg/kg/min, to achieve the desired blood pressure goal. The total mg amount of epinephrine in a pharmaceutical premix formulation described herein can range in part based on the volume of the flexible container containing the formulation, e.g., about 4 mg, about 8 mg, and about 16 mg per bag. For example, each ready to use bag containing the formulation disclosed herein could represent approximately a day's supply of medication which is appropriate for providing hemodynamic support in septic shock associated hypotension in adult patients. In some embodiments, the pharmaceutical premix formulation provided herein comprises about 4 mg of epinephrine. In some embodiments, the pharmaceutical premix formulation provided herein comprises about 8 mg of epinephrine. In some other embodiments, the pharmaceutical premix formulation provided herein comprises about 16 mg of epinephrine. The dose is variable based on patient weight and patient response.

In certain embodiments, the pharmaceutical premix formulation comprises epinephrine, sodium chloride, edetate disodium dihydrate, and sodium metabisulfite. The pharmaceutical premix formulation may contain edetate disodium dihydrate in a range from about 0.01 to about 0.3 mg/ml. In some embodiments, the pharmaceutical premix formulation comprises edetate disodium dihydrate in a range from about 0.025 to about 0.035 mg/ml. In some embodiments, the pharmaceutical premix formulation disodium edetate dihydrate in a range from about 0.030 to about 0.035 mg/ml. In one embodiment, a pharmaceutical premix formulation comprises about 0.032 mg/ml disodium edetate dihydrate.

In some embodiments, the pharmaceutical premix formulation comprises sodium metabisulfite (SMBS). Sodium metabisulfite (Na₂S₂O₅) is a compound having antioxidant, preservative, and antimicrobial activities. Without wishing to be bound by theory, it is believed that sodium metabisulfite suppresses or reduces the rate of oxidative degradation of epinephrine, for example, by reacting with oxygen, reactive radical species, and the like. The concentration of sodium metabisulfite present in the formulation may vary. For example, in some embodiments, the pharmaceutical premix formulation may contain from about 0.03 to about 0.07 mg/ml sodium metabisulfite, such as about 0.03, about 0.04, about 0.05, about 0.06, or about 0.07 mg/ml of sodium metabisulfite. The concentration of sodium metabisulfite present in the formulation may also be expressed in terms of molarity (i.e., mM). In an acidic environment, metabisulfite ions react with water and protons to provide sulfite ions (SO₃²⁻), with each millimole of sodium metabisulfite providing two millimoles of sulfite ions (two sulfite equivalents). In some embodiments, the pharmaceutical premix formulation comprises from about 0.316 millimoles of sulfite per liter (about 0.316 mM sulfite) to about 0.738 millimoles of sulfite per liter (about 0.738 mM sulfite), corresponding to about 0.03 to about 0.07 mg/ml sodium metabisulfite. In some embodiments, the pharmaceutical premix formulation comprises about 0.316 mM sulfite. In some embodiments, the pharmaceutical premix formulation comprises about 0.527 mM sulfite. In some embodiments, the pharmaceutical premix formulation comprises about 0.712 mM sulfite.

In some embodiments, the concentration of sulfite present in the pharmaceutical premix formulation may be expressed as a molar ratio to the concentration of epinephrine present in the pharmaceutical premix formulation. In some embodiments, the molar ratio of epinephrine to sulfite is in a range from about 0.07 to about 1.2, or from about 0.07 to about 0.66. In some embodiments, the molar ratio of epinephrine to sulfite is from about 0.07, about 0.08, about 0.09, or about 0.10, to about 0.15, about 0.2, about 0.25, about 0.3, about 0.35, about 0.4, about 0.45, about 0.5, about 0.55, about 0.6, or about 0.65. In some embodiments, the molar ratio of epinephrine to sulfite is 0.07. In some embodiments, the molar ratio of epinephrine to sulfite is 0.17. In some embodiments, the molar ratio of epinephrine to sulfite is 0.33. In some embodiments, the molar ratio of epinephrine to sulfite is 0.52. In some embodiments, the molar ratio of epinephrine to sulfite is 0.66.

For example, the pharmaceutical premix formulation may contain about 0.01 to about 0.3 mg/ml edetate disodium dihydrate and/or about 0.03 to about 0.07 mg/ml sodium metabisulfite (about 0.316 mM sulfite to about 0.738 mM sulfite).

In certain embodiments, the pharmaceutical premix formulation contains about 0.032 mg/ml edetate disodium dihydrate and about 0.05 mg/ml sodium metabisulfite (0.527 mM sulfite).

Examples of stable premix epinephrine formulations are provided in the table below:

| Ingredients | Amounts | | | | | |
|---|---|---|---|---|---|---|
| Epinephrine Bitartrate | 16 | mcg/mL | 32 | mcg/mL | 64 | mcg/mL |
| eq. to Epinephrine | | | | | | |
| Sodium Chloride | 9.0 | mg/mL | 9.0 | mg/mL | 9.0 | mg/mL |
| Sodium Metabisulfite | 0.05 | mg/mL | 0.05 | mg/mL | 0.05 | mg/mL |
| Disodium Edetate | 0.032 | mg/mL | 0.032 | mg/mL | 0.032 | mg/mL |
| Dihydrate (EDTA) | | | | | | |

| | | | |
|---|---|---|---|
| Sodium hydroxide | QS to | QS to | QS to |
| | adjust pH | adjust pH | adjust pH |
| Hydrochloric Acid | QS to | QS to | QS to |
| | adjust pH | adjust pH | adjust pH |
| Water for Injection | QS to 1 mL | QS to 1 mL | QS to 1 mL |

The pH of the pharmaceutical premix formulation described herein is a pH that maintains the stability of epinephrine. The pH of the pharmaceutical premix formulation is in the range of about 2.0 to about 6.0, such as, for example, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9 and about 6.0 (ranges including the numbers described herein are also contemplated, about 3.8 to about 5.0). In certain embodiments, the pharmaceutical premix formulation has a pH of about 2 to about 6. In some embodiments, pharmaceutical premix formulation has a pH of about 2.2 to about 5.5.

The pH of the solution may be adjusted by use of a pH adjusting agent, and optionally, if needed a buffer may be used to maintain the pH in the said range. The pH adjusting agent that may be used includes, but is not limited to, sodium hydroxide, potassium hydroxide, hydrochloric acid, sulphuric acid, acetic acid, sodium acetate, tartaric acid, and the like, and mixtures thereof. In one embodiment, the pH adjusting agent is sodium hydroxide, hydrochloric acid, or a combination thereof.

In some embodiments, a pharmaceutical premix formulation provided herein contains epinephrine, sodium chloride, edetate disodium dihydrate, and sodium metabisulfite in water and has a pH of about 2.2 to about 5.5. In one embodiment, the pharmaceutical premix formulation comprises from about 10 mcg/ml to about 70 mcg/ml epinephrine. In one embodiment, the pharmaceutical premix formulation comprises from about 8.4 mg/ml to about 9.4 mg/ml sodium chloride. In one embodiment, the pharmaceutical premix formulation comprises from about 0.01 to about 0.3 mg/ml edetate disodium dihydrate and/or from about 0.03 to about 0.07 mg/ml sodium metabisulfite.

In some embodiments, a pharmaceutical premix formulation provided herein contains about 16 mcg/ml epinephrine, about 0.9% sodium chloride, about 0.032 mg/ml edetate disodium dihydrate, and about 0.05 mg/ml sodium metabisulfite in water for injection and has a pH from about 2.2 to about 5.5. In such embodiments, the molar ratio of epinephrine to sulfite is 0.17.

In some embodiments, a pharmaceutical premix formulation provided herein contains about 32 mcg/ml epinephrine, about 0.9% sodium chloride, about 0.032 mg/ml edetate disodium dihydrate, and about 0.05 mg/ml sodium metabisulfite, in water for injection and has a pH from about 2.2 to about 5.5. In such embodiments, the molar ratio of epinephrine to sulfite is 0.33.

In some embodiments, a pharmaceutical premix formulation provided herein contains about 64 mcg/ml epinephrine, about 0.9% sodium chloride, about 0.032 mg/ml edetate disodium dihydrate, and about 0.05 mg/ml sodium metabisulfite, in water for injection, where the formulation has a pH from about 2.2 to about 5.5. In such embodiments, the molar ratio of epinephrine to sulfite is 0.66.

In some embodiments, the ready-to-use, sterile, stable pharmaceutical premix formulation is free of preservatives.

The pharmaceutical premix formulation of the disclosure may be characterized according to stability, such as long-term stability to storage. For example, in some embodiments, the pharmaceutical premix formulation is stable for at least about 3 months or at least about 6 months of storage at about 25±2° Celsius. In certain embodiments, the pharmaceutical premix formulation described herein is stable for about 12 months or more at about 25±2° Celsius. In some embodiments, the pharmaceutical premix formulation is stable for at least about 3 months or at least about 6 months of storage at about 40±2° C.

In some embodiments, stability of a pharmaceutical premix formulation is determined by a certain minimal level of degradant(s) over a period of time. For example, in one embodiment the pharmaceutical premix formulation has an initial concentration of epinephrine that is essentially maintained over a storage period; for example, about 1% or less of the epinephrine initially present degrades upon storage, such as less than about 0.5%, less than about 0.1%, or less than about 0.01%. The concentration of epinephrine present after storage may be determined according to methods known in the art. In some embodiments, a stable epinephrine formulation comprises a minimal amount of an impurity(ies), such as degradation products of epinephrine, that can be formed during storage. For example, in some embodiments, the pharmaceutical premix formulation contains a total of less than about 2% of impurities as measured by high performance liquid chromatography using UV absorption detection (HPLC/UV) monitoring at the wavelengths of 254 and 272 nm.

An example of an impurity that can be measured in the pharmaceutical premix formulation to determine overall stability is D-epinephrine (the (+)-enantiomer of epinephrine). In certain embodiments, the pharmaceutical premix formulation contains no more than about 5.5% D-epinephrine. In certain embodiments, the amount of D-epinephrine can be measured by HPLC over a determined time, e.g., about 3 months at about 25 degrees Celsius, about 6 months at about 25 degrees Celsius, about 9 months at about 25 degrees Celsius, or about 12 months at about 25 degrees Celsius. Other examples of amounts of D-epinephrine suggestive of stability are described in Tables 1 to 23 below.

Another example of a degradant that can be measured in the pharmaceutical premix formulation to determine stability is Impurity F. In certain embodiments, the pharmaceutical premix formulation contains no more than about 7.5% Impurity F. In certain embodiments, the amount of Impurity F can be measured by HPLC over a determined time, e.g., about 3 months at about 25 degrees Celsius, about 6 months at about 25 degrees Celsius, about 9 months at about 25 degrees Celsius, or about 12 months at about 25 degrees Celsius. Other examples of amounts of Impurity F suggestive of stability are described in Tables 1 to 23 below. In one embodiment, the pharmaceutical premix formulation contains no more than about 7.5% Impurity F as determined by HPLC. In one embodiment, the pharmaceutical premix formulation contains no more than about 7% Impurity F as determined by HPLC. In one embodiment, the pharmaceutical premix formulation contains no more than about 6.5% Impurity F as determined by HPLC. In one embodiment, the pharmaceutical premix formulation contains no more than about 6% Impurity F as determined by HPLC. In one embodiment, the pharmaceutical premix formulation contains no more than about 5.5% Impurity F as determined by HPLC.

In some embodiments, the pharmaceutical premix formulation is substantially colorless and contains at least about 90 wt. % of the epinephrine in the formulation prior to storage after at least about six months of storage at about 25±2° Celsius. In some embodiments, the pharmaceutical premix formulation is substantially colorless and contains at least 90 wt. % of the epinephrine in the formulation prior to storage after at least about three months of storage at about 40±2° Celsius.

As described above, impurities may be formed via degradation of one or more components of the pharmaceutical premix formulation. Sources of degradation include, but are not limited to, oxidation, racemization, sulfite addition, visible light, ultraviolet light, moisture, heat, changes in pH, and composition component interactions. Unless indicated otherwise, the percentages of impurities expressed herein are expressed as % w/w of the active agent. In some embodiments, the pharmaceutical premix formulation may have no more than about 20% of total impurities after a certain period of shelf life, more preferably no more than about 19.5%, more preferably no more than about 19%, more preferably no more than about 18.5%, preferably no more than about 18%, more preferably no more than about 17.5%, more preferably no more than about 17%, more preferably no more than about 16.5%, more preferably no more than about 16%, more preferably no more than about 15.5%, more preferably no more than about 15%, more preferably no more than about 14.5%, more preferably no more than about 14%, more preferably no more than about 13.5%, more preferably no more than about 13%, more preferably no more than about 12.5%, more preferably no more than about 12%, more preferably no more than about 11.5%, more preferably no more than about 11%, more preferably no more than about 10.5%, more preferably no more than about 10%, more preferably no more than about 9.5%, more preferably no more than about 9%, more preferably no more than about 8.5%, more preferably no more than about 8%, more preferably no more than about 7.5%, more preferably no more than about 7%, more preferably no more than about 6.5%, more preferably no more than about 6%, more preferably no more than about 5.5%, and most preferably no more than about 5%. In some embodiments, the pharmaceutical premix formulation contains less than about 1%, less than about 0.5%, or less than about 0.2% of impurities. In certain embodiments, impurities in the formulation do not include impurities Impurity F and D-epinephrine.

In one embodiment, the pharmaceutical premix formulation comprises a low level of epinephrine-related impurities. In certain embodiments, epinephrine-related impurities refer to the degradation product D-epinephrine. In one embodiment, the pharmaceutical premix formulation contains less than about 1%, less than about 0.5%, or less than about 0.2% of epinephrine-related impurities as determined by HPLC/UV. In some embodiments, the concentration of D-epinephrine in the pharmaceutical premix formulation after a certain period of shelf life may be no more than about 9.5%, preferably no more than about 9%, more preferably no more than about 8.5%, more preferably no more than about 8%, more preferably no more than about 7.5%, more preferably no more than about 7%, more preferably no more than about 6.5%, more preferably no more than about 6%, more preferably no more than about 5.5%, more preferably no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%, and most preferably no more than about 0.5% In some embodiments, less than about 10% of the epinephrine in the pharmaceutical premix formulation is D-epinephrine after at least about six months of storage at about 25±2° C. In some embodiments, less than 10% of the epinephrine in the pharmaceutical premix formulation is D-epinephrine after at least about six months of storage at about 30±2° Celsius. In some embodiments, less than about 10% of the epinephrine in the pharmaceutical premix formulation is D-epinephrine after at least about three months of storage at about 40±2° Celsius.

The pharmaceutical premix formulation of the disclosure has long term stability, e.g., the formulation is stable for at least about 6 months at about 25 degrees Celsius or stable for about 12 months at about 25 degrees Celsius. In other embodiments, the pharmaceutical premix formulation is stable for about 3 months at about 25 degrees Celsius.

Stability of the pharmaceutical premix formulation is achieved without the use of a preservative. Thus, in certain embodiments, a pharmaceutical premix formulation of the disclosure comprising epinephrine is essentially free of a preservative. Examples of preservatives include, but are not limited to, sodium benzoate, EDTA, sorbic acid, and parabens.

In certain embodiments, provided herein is a room temperature stable, ready to use premix formulation of epinephrine at a concentration of about 16 μg/ml, about 9.0 mg/ml sodium chloride (in a flexible container such as a 250 mL VIAFLO Container Closure System), about 0.05 mg/ml sodium metabisulfite, and about 0.032 mg/ml edetate disodium dihydrate, and has pH from about 2.2 to about 5.0, in diluted form, which can be used for as a continuous infusion depending on dosage requirement. In certain embodiments, provided herein is a room temperature stable, ready to use premix formulation of epinephrine at a concentration of about 32 μg/ml, about 9.0 mg/ml sodium chloride (in a flexible container such as a 250 mL VIAFLO Container Closure System), about 0.05 mg/ml sodium metabisulfite, and about 0.032 mg/ml edetate disodium dihydrate, and has a pH from about 2.2 to about 5.0, in diluted form, which can be used for as a continuous infusion depending on dosage requirement. In certain embodiments, provided herein is a room temperature stable, ready to use premix formulation of epinephrine at a concentration of about 64 μg/ml, about 9.0 mg/ml sodium chloride (in a flexible container such as a 250 mL VIAFLO Container Closure System), about 0.05 mg/ml sodium metabisulfite, and about 0.032 mg/ml edetate disodium dihydrate, and has a pH from about 2.2 to about 5.0, in diluted form, which can be used for as a continuous infusion depending on dosage requirement. In a preferred embodiment, the pharmaceutical premix formulation is not diluted prior to intravenous infusion into a subject (e.g., human patient).

An epinephrine pharmaceutical premix formulation described herein can be contained in a flexible container. In some embodiments, the flexible container described herein consist essentially of the epinephrine pharmaceutical premix formulation provided herein. The volume capacity of the flexible container can range, for example, from about 50 mL to about 1000 mL. In certain embodiments, the volume capacity of each flexible container may range from about 50 ml to about 500 ml, such as for example about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440 or about 450 ml, more preferably from about 50 ml to about 250 ml. According to some embodiments, the container (e.g., infusion bag) can accommodate a volume from about 100 ml to about 250 ml, preferably about 250 ml. In certain embodiments, the volume of the flexible container is about 50 ml. In certain embodiments, the volume of the flexible container is about 100 ml. In certain embodiments, the volume of the flexible container is about 250 ml. In certain embodiments, the volume of the flexible container is about 500 ml. In certain embodiments, the volume of the flexible container is about 1000 ml. The flexible container, e.g., a plastic bag, may be suitable for intravenous infusion of the formulation to a human subject. In some embodiments, the ready-to-use, sterile, stable premix formulation of epinephrine filled in the flexible container comprises a concentration in the range of about 16 μg/ml to about 64 μg/ml of epinephrine. In one embodiment, the ready-to-use, sterile, stable premix formulation of epinephrine filled in the flexible container comprises a concentration of about 16 μg/ml of epinephrine. In another embodiment, the ready-to-use, sterile, stable premix formulation of epinephrine filled in the flexible container comprises a concentration of about 32 μg/ml of epinephrine. In yet another embodiment, the ready-to-use, sterile, stable premix formulation of epinephrine filled in the flexible container comprises a concentration of about 64 μg/ml of epinephrine.

The material of the flexible container can be a plastic, e.g., the container may include polypropylene (PP), polyamide (PA), and polyethylene (PE). In certain embodiments, the flexible container is a VIAFLO container, which is a bag composed of polyolefin/polyamide co-extruded plastic. The premix formulation may be administered to a human patient intravenously via an infusion tube connected to the flexible container. A flexible container also provides better control over a rigid container for safety purposes. By avoiding dilution, where the sterile premix formulation is ready to use, contamination and/or medication error can be avoided. In some embodiments, the flexible container is part of an intravenous hook system such that the intravenous hook comprises the flexible container with the stable, ready-to-use pharmaceutical premix formulation of epinephrine.

The flexible container comprising the ready-to-use, sterile, stable premix formulation of epinephrine can be terminally sterilized without compromising the stability of epinephrine. The flexible container is capable of maintaining the stability of the solution after terminal sterilization by autoclaving and upon storage at room temperature for a period of at least about 6 months. It was also found that epinephrine solution when stored in a VIAFLO container remained stable upon long term storage with no signs of any visible particles, and impurities also remained under pharmaceutically acceptable range.

The ready-to-use epinephrine formulation of the invention may be packaged in any suitable primary container known in the art including but not limited to vials, syringes, bags, bottles and ampules presentations. Containers may be fabricated from glass or from polymeric materials. The size of the primary container typically ranges from about 1 nil to about 500 ml. Ready-to-use epinephrine formulations disclosed herein may be filled into bags, bottles, ampules, or vials with sizes generally between about 1 ml and about 500 ml, for example, about 50 mL or about 100 mL bags. Suitable primary containers include flexible bags as disclosed in US 2008/0249499, which is hereby incorporated by reference in its entirety.

Other flexible bags may be used. Preferred flexible bag primary containers may be free of PVC, such as those disclosed in U.S. Pat. Nos. 5,849,843 and 5,998,019, which are hereby incorporated by reference in their entirety. Suitable flexible polymeric primary containers include but are not limited to GALAXY IV containers (Baxter International Inc.), VIAFLO containers (Baxter International Inc.), and INTRAVIA containers (Baxter International Inc.). In one embodiment, a flexible container used to house the premix epinephrine formulation is a flexible plastic container fabricated from a multilayer sheeting composed of polypropylene (PP), polyamide (PA) and polyethylene (PE). In one embodiment, the inner layer of the flexible container is made of polyethylene and is in contact with the phenylephrine HCl premix pharmaceutical formulation.

Provided herein is also a system containing the pharmaceutical premix epinephrine formulation described herein and an oxygen scavenger. The oxygen scavenger may be in the form of an oxygen absorbing sachet or a polyethylene container. The oxygen scavenger may, in certain embodiments, contain micronized iron, where, e.g., the iron in the oxygen absorbing sachet reacts with the oxygen in the headspace environment of the container to keep the oxygen content low within the system. Preferably the system is a container closure system, where the oxygen scavenger is outside of the container holding the pharmaceutical premix epinephrine formulation, such that the oxygen scavenger acts to stabilize the pharmaceutical premix epinephrine formulation by absorbing oxygen surrounding the container holding the formulation. Air (oxygen) can induce degradation via oxidation. Degradation can be minimized by filling the container as full as possible, thereby decreasing the headspace, or by replacing the headspace with nitrogen. The oxygen scavenger may be in the form of a sachet made from polyethylene materials, which contains an oxygen absorbing mixture composed primarily from micronized iron.

The system described herein may also contain a component which is photosensitive and prevents light from contacting the pharmaceutical premix epinephrine formulation. The effects of light can be minimized by packaging products (or providing a system) in light-resistant containers. For example, the system may contain a pouch which goes over the pharmaceutical premix epinephrine formulation to prevent light from contacting the formulation. The overpouch may be made of aluminum foil and/or an amber plastic overwrap.

The primary container may he disposed within and enclosed by a secondary container. The secondary container may be an overpouch container. Overpouches are flexible containers that can be used as secondary containers in the packaged, sealed container systems disclosed herein to store, protect, and transport, the primary containers containing a formulation comprising an oxygen-sensitive pharmaceutical compound such as epinephrine therein. Generally, overpouches are provided by a first flexible sheet layer, an opposing second flexible sheet layer, and a seal disposed along a common peripheral edge of the first and second flexible sheet layers. Preferably, the secondary overpouch container should he optically transparent to enable visual inspection of the primary container and any other contents within the overpouch.

it is also desirable for the overpouch container to be capable of ithstanding autoclaving or other terminal sterilization process without causing the medical component therein to shrink/wrinkle and without becoming discolored and/or adhered to the medical component.

The overpouch container may he an aluminum overpouch, a light absorbing polymeric overpouch, or a similar barrier structure. In one embodiment, the primary container is in fluid communication with any other contents of the overpouch secondary container.

In one embodiment, the overpouch secondary container comprises a first flexible sheet layer comprising an amber transparent film and a second flexible sheet layer comprising an opaque aluminum laminated foil. The first flexible sheet layer of may be an amber transparent multilayer film comprising a PET (Polyethylene terephthalate)/PA/PP laminate to allow the contents within the secondary container, for example, any labeling on the primary container to be seen. The second flexible sheet layer may be an opaque laminated foil comprising a PET/PA/Aluminum foil/PP laminate. A seal is disposed along a common peripheral edge of the first and second flexible sheets.

In another embodiment, the overpouch secondary container comprises a first flexible single layer sheet layer comprising a polymeric blend of a high density polyethylene and a surface enhancing polymer, an opposing second flexible single layer sheet layer comprising a polymeric blend of a high density polyethylene and a surface enhancing polymer of an ethylene propylene diene terpolymer dispersed in a polyolefin matrix, and a seal disposed along a common peripheral edge of the first and second flexible sheets as disclosed in US 2006/0240204, which is hereby incorporated by reference in its entirety.

An oxygen scavenger is also disposed within and enclosed by the overpouch secondary container. A sachet located adjacent to the primary container and disposed within the secondary overpouch container may include the oxygen scavenger. The sachet (i.e., the bag) itself is porous and may comprise polyethylene materials. The oxygen scavenger may comprise iron powder, iron oxide powder, or a mixture thereof, for example, micronized iron. Other known oxygen scavengers may also be used. The oxygen scavenger is primarily included to absorb small amounts of oxygen that permeate through the secondary container during the shelf life of the drug product.

Thus, despite the primary and secondary containers being sealed, the fluid contents of the primary container may be considered to be in fluid communication with the contents of the secondary container, including the oxygen scavenger. Thus, the oxygen scavenger can be considered to be in fluid communication with the formulation in the primary container.

Generally, epinephrine injection is available as a concentrated solution which needs to be diluted before administration as an intravenous bolus or continuous intravenous infusion to achieve the desired concentration. The concentrated epinephrine formulation, for example, generally is diluted with 0.9% sodium chloride or 5% dextrose in water. Epinephrine is mainly administered by intramuscular, subcutaneous, and intravenous routes. For intravenous infusion it is diluted to a concentration of about 1.0 μg/ml with 5% Dextrose or 5% Dextrose and sodium chloride solution as per approved product information. Once the diluted solutions are prepared, they are not typically stored for more than 4 hours at room temperature or 24 hours under refrigerated conditions. To provide hemodynamic support in septic shock associated hypotension in adult patients, the suggested dosing infusion rate of intravenously administered epinephrine is about 0.05 to about 2 mcg/kg/min and is titrated to achieve a desired mean arterial pressure (MAP). The dosage may be adjusted periodically, such as every 10-15 minutes, in increments of 0.05 to 0.2 mcg/kg/min, to achieve the desired blood pressure goal.

The formulations and the flexible container disclosed herein provide advantages over those other epinephrine formulations known in the art. For example, a premix liquid formulation, like those disclosed herein, provides a certain dose amount (e.g., an IV bolus/Infusion) that is readily available to the patient without a need to first dilute the epinephrine formulation. By providing a premix liquid formulation, which does not require dilution, the risk of contamination and compounding, or medication error associated therewith, is essentially eliminated. Furthermore, the flexible container of the present disclosure provides a means of direct intravenous administration of the sterile, stable formulation of epinephrine or a pharmaceutically acceptable salt thereof, to the patient through the infusion container, using the outlet port. Further, the formulation disclosed herein reduces time needed by medical experts to dilute and prepare epinephrine for administration, and also reduces medical waste.

Importantly, the formulation described herein, which can be contained in a flexible container such as a plastic bag suitable for intravenous infusion, are ready to use for delivery of the epinephrine to the patient, i.e., there is no need to dilute the epinephrine and the premix formulation contained within the flexible container can be administered to the patient without a mixing and/or dilution step. In one embodiment, the system described herein (i.e., premix formulation in a flexible container, such as a VIAFLO plastic bag), provides improved safety for the patient by providing better control in contrast to rigid containers used to mix and dilute epinephrine to achieve a suitable concentration for administering an accurate dose.

In some aspects, there are provided methods of using the stable, ready to use premix formulation of epinephrine. Disclosed herein are methods of treating a human subject having a septic shock associated hypotension. In certain embodiments, the stable, ready to use premix formulation of epinephrine is administered to a human subject having a septic shock associated hypotension, wherein administration of the formulation reduces, alleviates, or eliminates the septic shock associated hypotension.

In one embodiment, a method of treating a human subject having a septic shock associated hypotension, is described herein. The method comprises the steps of obtaining the flexible container provided herein comprising the pharmaceutical premix formulation described above, placing the flexible container on a hook or means for suspending the flexible container, and intravenously administering the ready to use premix formulation of epinephrine into the human subject.

In another embodiment, a method of treating a septic shock associated hypotension or anaphylaxis in a human subject in need thereof is described herein. The method comprises intravenously administering to the human subject a pharmaceutical premix formulation comprising about 10 μg/ml to about 70 μg/ml, such as for example about 10, about 15, about 20, about 25, about 30, about 35, about 40, about 45, about 50, about 55, about 60, about 65 or about 70 μg/ml, more preferably from about 16 μg/ml to about 64 μg/ml, of epinephrine. In some embodiments, the liquid formulation in the method described above further comprises about 0.8% to about 0.9% sodium chloride. In some embodiments, the liquid formulation in the method described above further comprises about 0.025 mg/ml to about 0.1 mg/ml sodium metabisulfite. In one embodiment, the method comprises intravenously administering a pharmaceutical premix formulation comprising about 16 μg/ml epinephrine, about 0.9% sodium chloride and about 0.05 mg/ml sodium metabisulfite, to the human subject. In another embodiment, the method comprises intravenously administering a pharmaceutical premix formulation comprising about 32 μg/ml epinephrine, about 0.9% sodium chloride and about 0.05 mg/ml sodium metabisulfite, to the human subject. In yet another embodiment, the method comprises intravenously administering a pharmaceutical premix formulation comprising about 64 μg/ml epinephrine, about 0.9% sodium chloride and about 0.05 mg/ml sodium metabisulfite, to the human subject

In one embodiment, the method comprises intravenously administering a pharmaceutical premix formulation comprising about 4 mg of epinephrine at a concentration of about 16 μg/ml, about 9.0 mg/ml sodium chloride, and about 0.05 mg/ml sodium metabisulfite, and having a pH from about 2.2 to about 5.0, to the human subject. In one embodiment, the method comprises intravenously administering a pharmaceutical premix formulation comprising about 8 mg of epinephrine at a concentration of about 32 μg/ml, about 9.0 mg/ml sodium chloride, and about 0.05 mg/ml sodium metabisulfite, and having a pH from about 2.2 to about 5.0. In one embodiment, the method comprises intravenously administering a pharmaceutical premix formulation comprising about 16 mg of epinephrine at a concentration of about 64 μg/ml, about 9.0 mg/ml sodium chloride, and about 0.05 mg/ml sodium metabisulfite, and having a pH from about 2.2 to about 5.0. In the embodiments of the methods described above, the formulation is an aqueous, premix formulation that is not diluted prior to administration to the human subject.

In many embodiments of the methods described above, the pharmaceutical premix formulation comprising epinephrine is stored and infused from a flexible container such as a 100 or 250 mL VIAFLO Container Closure System, which can be used for continuous infusion depending on dosage requirement. In some embodiments, the VIAFLO Container is part of an intravenous hook system such that the intravenous hook comprises the VIAFLO Container with the stable, ready-to-use pharmaceutical premix formulation of epinephrine.

In many embodiments of the methods described above, the pharmaceutical premix formulation comprising epinephrine is essentially free of a preservative.

In many embodiments of the methods described above, the pharmaceutical premix formulation comprising epinephrine is stable for at least about 6 months at about 25 degrees Celsius. In some embodiments of the methods, the formulation is stable for at least about 6 months at about 30 degrees Celsius. In other embodiments of the methods, the formulation is stable for about 1 year at about 30 degrees Celsius. In a specific embodiment of the method, the formulation is stable for at least about 3 months at about 30 degrees Celsius. In some embodiments of the methods, the formulation is stable for at least about 63 months at about 40 degrees Celsius. In other embodiments of the methods, the formulation is stable for at least about 3 months at about 40 degrees Celsius. In a specific embodiment of the method, the formulation is stable for at least about 1 month at about 40 degrees Celsius.

In many embodiments of the methods described herein, epinephrine is administered as a continuous intravenous dose to a human subject at an infusion rate of between about 0.05 μg/kg/hr and about 2 μg/kg/hr, or between about 0.05 μg/kg/hr and about 0.075 μg/kg/hr, or between about 0.05 μg/kg/hr and about 0.1 μg/kg/hr, or between about 0.05 μg/kg/hr and about 0.125 μg/kg/hr, or between about 0.05 μg/kg/hr and about 0.15 μg/kg/hr, or between about 0.05 μg/kg/hr and about 0.175 μg/kg/hr, or between about 0.05 μg/kg/hr and about 0.2 μg/kg/hr. The dosage may be adjusted periodically, such as every 10-15 minutes, in increments of about 0.05 to about 0.2 mcg/kg/min, to achieve the desired blood pressure goal.

In many embodiments of the methods described herein, the epinephrine is administered as a continuous intravenous dose for a period of time of between about 1 and about 10 minutes, or between about 1 and about 20 minutes, or between about 1 and about 30 minutes, or between about 1 and about 2 hours, or between about 1 and about 3 hours, or between about 1 and about 4 hours, or between about 1 and about 5 hours, or between about 1 and about 6 hours, or between about 1 and about 7 hours, or between about 1 and about 8 hours, or between about 1 and about 9 hours, or between about 1 and about 10 hours, or between about 1 and about 11 hours, or between about 1 and about 12 hours, or between about 1 and about 13 hours, or between about 1 and about 14 hours, or between about 1 and about 15 hours, or between about 1 and about 16 hours, or between about 1 and about 17 hours, or between about 1 and about 18 hours, or between about 1 and about 19 hours, or between about 1 and about 20 hours, or between about 1 and about 21 hours, or between about 1 and about 22 hours, or between about 1 and about 23 hours, or between about 1 and about 24 hours.

In some embodiments, the human subject treated with the pharmaceutical premix formulation disclosed herein, is critically ill. In one embodiment, the human subject suffers from one or more medical conditions. In certain embodiments, the medical condition is a lung problem, brain problem, heart problem, liver problem, kidney problem, eye or ear problem, gastrointestinal problem, or skin problem.

In certain embodiments, the pharmaceutical premix formulation disclosed herein may be administered to a human subject to rapidly to improve breathing, stimulate the heart, raise dropping blood pressure, reverse hives, and reduce swelling of the face, lips, and throat. In some other embodiments, the pharmaceutical premix formulation disclosed herein may be administered to a human subject as an emergency treatment of allergic reactions (Type 1), including anaphylaxis, induction, and maintenance of mydriasis during intraocular surgery, treatment of bronchospasm, sensitivity reactions, cardiac arrhythmias, GI and renal hemorrhage, superficial bleeding, premature labor, hypoglycemia, and cardiogenic, hemorrhagic, and traumatic shock. In some other embodiments, the pharmaceutical premix formulation disclosed herein may be administered to a human subject to increase blood flow in ACLS during CPR, as an adjunct to local anesthesia, and for radiographic uses. In some other embodiments, the pharmaceutical premix formulation disclosed herein may be administered to a human subject to lower intraocular pressure, for example, in the treatment of glaucoma.

In certain embodiments, the pharmaceutical premix formulation disclosed herein do not include any other active ingredient, or therapeutic agent, other than epinephrine.

The total mg of epinephrine in the premix formulation described herein (also described in terms of the amount of premix formulation in a flexible container) can be, for example about 2 to about 20 mg. In one embodiment, a flexible container is a ready to use bag, e.g., a container closure system such as VIAFLO, and contains about 4 mg of epinephrine in 250 ml, so the flexible container comprises approximately a day's supply of medication which is appropriate for the continuous infusion protocol to treat septic shock associated with hypotension or anaphylaxis. In another embodiment, a flexible container is a ready to use bag, e.g., a container closure system such as VIAFLO, and contains about 8 mg of epinephrine in 250 ml, so the flexible container comprises approximately a day's supply of medication which is appropriate for the continuous infusion protocol to treat septic shock associated with hypotension or anaphylaxis. a flexible container is a ready to use bag, e.g., a container closure system such as VIAFLO, and contains about 16 mg of epinephrine in 250 ml, so the flexible container comprises approximately a day's supply of medication which is appropriate for the continuous infusion protocol to treat septic shock associated with hypotension or anaphylaxis

In certain embodiments, the dose of epinephrine that is administered to the human subject will be variable based on patient weight and patient response. In some embodiments, the dose of epinephrine and the infusion rate are selected from those in the table below.

| | Minimum Approved Dose | | Maximum Approved Dose | |
|---|---|---|---|---|
| | (0.05 mcg/kg/min) | | (2 mcg/kg/min) | |
| | Infusion | 24 h | Infusion | 24 h |
| Epinephrine | rate | infusion | rate | infusion |
| concentration | (mL/min) | volume (mL) | (mL/min) | volume (mL) |
| 1 mcg/mL | 3.5 | 5040 | 140 | 201600 |
| (concentration | | | | |
| recommended in USPI) | | | | |
| 4000 mcg Base/250 | 0.22 | 315 | 8.75 | 12600 |
| mL (16 mcg Base/mL | | | | |
| 8000 mcg Base/250 | 0.11 | 157.5 | 4.38 | 6300 |
| mL (32 mcg Base/mL) | | | | |
| 16000 mcg Base/250 | 0.05 | 78.8 | 2.19 | 3150 |
| mL (64 mcg Base/mL) | | | | |

In some embodiments, a dose of epinephrine is administered to the subject per day, e.g., about 2 to about 20 mg per day. In one embodiment, about 0.5 to about 4 mg of epinephrine is administered to a human subject, slowly or infused over several minutes. This dose may be repeated at 10-to-15-minute intervals until adequate blood pressure is achieved. In one embodiment, about 4 to about 8 mg of epinephrine is administered to a human subject in need thereof, e.g., a human subject experiencing anaphylactic shock. In another embodiment, about 8 to aboutl6 mg of epinephrine is administered to a human subject in need thereof, e.g., a human subject undergoing an extreme allergic reaction and has respiratory complications surgery, in a 24-hour period through continuous infusion. In another embodiment, about 0.05 to about 2 μg /kg/hr continuous infusion dose is administered to a human subject in need thereof, intravenously over 24 hours, and may be increased if there is no adequate response is achieved.

The formulation of epinephrine described herein may also contain a certain dose of the drug. For example, the premix formulation may contain about 40 to about 100 mg of epinephrine. A bag (or flexible container) containing the premix formulation is ready to use and does not require dilution prior to administration as the bag (or flexible container) provides the required dosage regimen (IV bolus/Infusion) to the patient based on the requirement.

In certain embodiments, pharmaceutical premix formulations include those described in the following table:

| Ingredients | Amounts | | | | | |
|---|---|---|---|---|---|---|
| Epinephrine Bitartrate | 16 | mcg/mL | 32 | mcg/mL | 64 | mcg/mL |
| eq. to Epinephrine | | | | | | |
| Base | | | | | | |
| Sodium Chloride | 9.0 | mg/mL | 9.0 | mg/mL | 9.0 | mg/mL |
| Sodium Metabisulfite | 0.05 | mg/mL | 0.05 | mg/mL | 0.05 | mg/mL |
| Edetate Disodium | 0.032 | mg/mL | 0.032 | mg/mL | 0.032 | mg/mL |
| Dihydrate (EDTA) | | | | | | |

| | | | |
|---|---|---|---|
| Sodium hydroxide | QS to | QS to | QS to |
| | adjust pH | adjust pH | adjust pH |
| Hydrochloric Acid | QS to | QS to | QS to |
| | adjust pH | adjust pH | adjust pH |
| Water for Injection | QS to 1 mL | QS to 1 mL | QS to 1 mL |

Thus, in one embodiment, an aqueous pharmaceutical formulation of the invention comprises water, about 16 mcg/mL epinephrine, about 9 mg/ml sodium chloride (about 0.9% sodium chloride), about 0.05 mg/mL sodium metabisulfite, and about 0.032 mg/ml sodium edetate dihydrate.

All methods described herein can be performed in any suitable order unless otherwise indicated herein or otherwise clearly contradicted by context. The use of any and all examples, or exemplary language (e.g., “such as”) provided herein, is intended merely to better illustrate the materials and methods and does not pose a limitation on the scope unless otherwise claimed. No language in the specification should be construed as indicating any non-claimed element as essential to the practice of the disclosed materials and methods.

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein.

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the technology. Thus, the appearances of phrases such as “in one or more embodiments,” “in certain embodiments,” “in one embodiment” or “in an embodiment” in various places throughout this specification are not necessarily referring to the same embodiment of the technology. Furthermore, the particular features, structures, materials, or characteristics may be combined in any suitable manner in one or more embodiments. Any ranges cited herein are inclusive.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

### EXAMPLES

The following examples support the concept of an aqueous, pharmaceutical premix epinephrine formulation which is ready to use and does not require dilution and is stable at room temperature. The following examples are included for illustrative purposes only and are not intended to limit the scope of the invention.

### Example 1

### Stability Testing of Epinephrine Premix Formulations Having Range of pH

The following example provides stability data for premix epinephrine formulations having a fixed concentration of epinephrine (16 μg/mL) and having a pH ranging from 3.4 to 6.0 (with sodium hydroxide and hydrochloric acid used as pH adjustors). All formulations contained 0.9% sodium chloride and 0.05 mg/mL sodium metabisulfite (SMBS) (.

For tables in Examples 1-2, the following is a summary of the specification limits of the descriptions provided therein:

| Table Description | Specification Limits |
|---|---|
| Description | A clear and colorless solution filled |
| | in a bag |
| Clarity of Solution | Clear solution essentially free from |
| | visible particulate matter. |
| Color and achromicity | Color of the solution should not be more |
| of solution | intense than purified water |
| pH | Between 3.0 and 5.0 (unless otherwise |
| | specified) |
| Each individual | Not More Than 0.2% |
| unspecified impurity | |
| Total degradation | Not More Than 1.3% |
| products | |
| Assay of L-Epinephrine | Between 90.0% and 110.0% of the label |
| by HPLC (“L-Epinephrine”) | claim of L-Epinephrine (Label claim: 16 |
| | mcg/mL) |
| | Between 90.0% and 110.0% of the label |
| | claim of L-Epinephrine (Label claim: 32 |
| | mcg/mL) |
| | Between 90.0% and 110.0% of the label |
| | claim of L-Epinephrine (Label claim: 64 |
| | mcg/mL) |
| Content of D-Epinephrine | Not More Than 5.5% |
| (S-Epinephrine) | |
| (“D-Epinephrine”) | |
| Impurity F (Imp F) | Not More Than 7.5% |
| (By HPLC) | |
| Total impurities | Not More Than 10.0% |
| (excluding Impurity F | |
| and D-Epinephrine) | |
| Assay of Sodium | Between 10.0% and 110.0% of the label |
| Metabisulfite (By HPLC) | claim of Sodium Metabisulfite (Label |
| | claims: 0.05 mg/mL) |
| Particulate Matter | NMT 6000 per container |
| For ≥10 μm | NMT 600 per container |
| For ≥25 μm | |

For all tables in Examples 1-2, the “Conditions” refer to the Time Point/Temperature/Relative Humidity for each formulation that was tested. 1M, 3M, or 6M, refer to a 1 month, 3 month, or 6 month time point, respectively. Temperature is referred to as 25 or 40 and refers to 25 degrees Celsius±2 degrees Celsius or 40 degrees Celsius±2 degrees Celsius, respectively. The relative humidity (RH) conditions associated with each temperature were not more than (NMT) 25% RH for 40 degrees Celsius and 40%±5% RH for 25 degrees Celsius. For example, in Table 1 below, “40 C/1 M” indicates testing conditions of 1 month at 40 degrees Celsius±2 degrees Celsius in NMT 25% RH. Impurity F (Imp F) is (1R)-1-(3,4-dihydroxyphenyl)-2-(methylamino) ethanesulfonic acid.

The data provided in Tables 1 to 5 below show that all parameters were found well within the specifications. Hence it is concluded that the product is stable when pH is maintained at a pH between about 3 and about 5, and beyond this limit the formulation fails. The results in Table 6 indicate that Imp F is out of the specification limit, and hence it is concluded that the product is not stable at pH 6.0.

TABLE 1
Epinephrine 16 mcg/mL at pH 3.4

| | Analytical Data | | | | | | |
|---|---|---|---|---|---|---|---|
| | | Color and | | | Assay | | |
| | | achromicity | Clarity | | L- | D- | |
| Conditions | Description | of solution | of solution | pH | Epinephrine | Epinephrine | SMBS |
| specification | A clear and | Color of the | Clear solution | Between | 90.0 to | NMT | 10.0 to |
| Limit | colorless | solution | essentially | 3.0 and | 110.0% | 5.5% | 110.0% |
| | solution | should not be | free from | 5.0 | | | |
| | filled | more intense | visible | | | | |
| | in a bag | than Purified | particles | | | | |
| | | water. | | | | | |
| Initial | Complies | Complies | Complies | 3.46 | 97.3 | 3.61 | 70.49 |
| 40 C./1 M | Complies | Complies | Complies | 3.61 | 95.22 | 4.22 | 60.46 |
| 40 C./2 M | Complies | Complies | Complies | 3.80 | 94.88 | 5.33 | 46.90 |
| 40 C./3 M | Complies | Complies | Complies | 3.83 | 95.31 | 6.56 | 42.4 |
| 30 C./3 M | Complies | Complies | Complies | 3.76 | 97.81 | 4.64 | 53.84 |
| 25 C./1 M | Complies | Complies | Complies | 3.56 | 96.48 | 3.44 | 68.59 |
| 25 C./2 M | Complies | Complies | Complies | 3.75 | 97.08 | 4.20 | 57.22 |
| 25 C./3 M | Complies | Complies | Complies | 3.74 | 98.24 | 4.35 | 56.98 |

| | Analytical Data | | | |
|---|---|---|---|---|
| | Related Substances | | | |
| | | | Total | |
| | | Each | impurity | |
| | | individual | (Excluding | |
| | Impurity | unspecified | Imp F and D- | Particulate |
| Conditions | F | impurity | Epinephrine) | matter |
| specification | NMT | NMT | NMT | For ≥ 10 μm: |
| Limit | 7.5% | 0.2% | 10.0% | NMT 6000 |
| | | | | particles per |
| | | | | container |
| | | | | For ≥ 25 μm: |
| | | | | NMT 600 |
| | | | | particles per |
| | | | | container |
| Initial | 1.080 | 0.041(RRT-1.09) | 0.041 | 10 μm: 133 |
| | | | | 25 μm: 13 |
| 40 C./1 M | 1.696 | 0.014(RRT-0.84) | 0.014 | 10 μm: 106.7 |
| | | | | 25 μm: 0.0 |
| 40 C./2 M | 4.063 | 0.041(RRT-0.84) | 0.119 | 10 μm: 33 |
| | | 0.026(RRT-0.95) | | 25 μm: 0 |
| | | 0.025(RRT-1.08) | | |
| 40 C./3 M | 3.182 | 0.047(RRT-0.95) | 0.137 | 10 μm: 53 |
| | | 0.042(RRT-0.85) | | 25 μm: 0.0 |
| | | 0.0199RRT-1.09) | | |
| | | 0.015(RRT-0.63) | | |
| 30 C./3 M | 1.689 | 0.044(RRT-0.95) | 0.134 | 10 μm :73 |
| | | 0.043(RRT-1.09) | | 25 μm: 0 |
| | | 0.037(RRT-0.85) | | |
| 25 C./1 M | 1.101 | ND | ND | 10 μm: 160.0 |
| | | | | 25 μm: 46.7 |
| 25 C./2 M | 1.404 | 0.042(RRT-1.08) | 0.117 | 10 μm: 40 |
| | | 0.037(RRT-0.84) | | 25 μm: 0 |
| | | 0.023(RRT-0.95) | | |
| 25 C./3 M | 1.348 | 0.054(RRT-1.09) | 0.162 | 10 μm: 493 |
| | | 0.045(RRT-0.84) | | 25 μm: 0 |
| | | 0.036(RRT-0.95) | | |
| | | 0.016(RRT-0.45) | | |
| | | 0.011(RRT-0.71) | | |

TABLE 2
Epinephrine 16 mcg/mL at pH 3.6

| | Analytical Data | | | | | | |
|---|---|---|---|---|---|---|---|
| | | Color and | | | Assay | | |
| | | achromicity | Clarity | | L- | D- | |
| Conditions | Description | of solution | of solution | pH | Epinephrine | Epinephrine | SMBS |
| specification | A clear and | Color of the | Clear solution | Between | 90.0 to | NMT | 10.0 to |
| Limit | colorless | solution | essentially | 3.0 and | 110.0% | 5.5% | 110.0% |
| | solution | should not be | free from | 5.0 | | | |
| | filled | more intense | visible | | | | |
| | in a bag | than Purified | particles | | | | |
| | | water. | | | | | |
| Initial | Complies | Complies | Complies | 3.66 | 96.17 | 3.22 | 77.42 |
| 40 C./1 M | Complies | Complies | Complies | 3.88 | 95.23 | 3.52 | 70.04 |
| 40 C./2 M | Complies | Complies | Complies | 3.91 | 93.16 | 3.84 | 60.69 |
| 40 C./3 M | Complies | Complies | Complies | 4.12 | 94.15 | 4.99 | 50.24 |
| 30 C./3 M | Complies | Complies | Complies | 3.97 | 96.72 | 3.96 | 63.83 |
| 25 C./1 M | Complies | Complies | Complies | 3.82 | 95.65 | 3.26 | 75.69 |
| 25 C./2 M | Complies | Complies | Complies | 3.84 | 94.72 | 3.15 | 68.97 |
| 25 C./3 M | Complies | Complies | Complies | 4.01 | 97.02 | 3.77 | 68.03 |

| | Analytical Data | | | |
|---|---|---|---|---|
| | Related Substances | | | |
| | | | Total | |
| | | Each | impurity | |
| | | individual | (Excluding | |
| | Impurity | unspecified | Imp F and D- | Particulate |
| Conditions | F | impurity | Epinephrine) | matter |
| specification | NMT | NMT | NMT | For ≥ 10 μm: |
| Limit | 7.5% | 0.2% | 10.0% | NMT 6000 |
| | | | | particles per |
| | | | | container |
| | | | | For ≥ 25 μm: |
| | | | | NMT 600 |
| | | | | particles per |
| | | | | container |
| Initial | 1.850 | ND | ND | 10 μm: 220 |
| | | | | 25 μm: 00 |
| 40 C./1 M | 2.790 | ND | ND | 10 μm: 53.3 |
| | | | | 25 μm: 13.3 |
| 40 C./2 M | 4.791 | 0.020(RRT-0.51) | 0.02 | NP |
| 40 C./3 M | 4.774 | 0.059(RRT-0.95) | 0.142 | 10 μm: 320 |
| | | 0.039(RRT-0.85) | | 25 μm: 13 |
| | | 0.019(RRT-1.09) | | |
| 30 C./3 M | 2.684 | 0.051(RRT-0.95) | 0.141 | 10 μm: 547 |
| | | 0.045(RRT-1.09) | | 25 μm: 20 |
| | | 0.033(RRT-0.85) | | |
| 25 C./1 M | 1.831 | ND | ND | 10 μm: 20.0 |
| | | | | 25 μm: 0.0 |
| 25 C./2 M | 3.726 | ND | ND | NP |
| 25 C./3 M | 2.239 | 0.047(RRT-0.95) | 0.144 | 10 μm: 620 |
| | | 0.037(RRT-1.09) | | 25 μm: 20 |
| | | 0.024(RRT-0.85) | | |
| | | 0.014(RRT-0.71) | | |
| | | 0.011(RRT-0.45) | | |

TABLE 3
Epinephrine 16 mcg/mL at pH 3.8

| | Analytical Data | | | | | | |
|---|---|---|---|---|---|---|---|
| | | Color and | | | Assay | | |
| | | achromicity | Clarity | | L- | D- | |
| Conditions | Description | of solution | of solution | pH | Epinephrine | Epinephrine | SMBS |
| specification | A clear and | Color of the | Clear solution | Between | 90.0 to | NMT | 10.0 to |
| Limit | colorless | solution | essentially | 3.0 and | 110.0% | 5.5% | 110.0% |
| | solution | should not be | free from | 5.0 | | | |
| | filled | more intense | visible | | | | |
| | in a bag | than Purified | particles | | | | |
| | | water. | | | | | |
| Initial | Complies | Complies | Complies | 4.14 | 98.22 | 1.77 | 87.75 |
| 40 C./1 M | Complies | Complies | Complies | 4.07 | 96.47 | 2.40 | 83.32 |
| 40 C./2 M | Complies | Complies | Complies | 4.12 | 94.47 | 2.45 | 82.88 |
| 40 C./3 M | Complies | Complies | Complies | 4.23 | 92.12 | 2.51 | 71.30 |
| 30 C./1 M | Complies | Complies | Complies | 3.98 | 97.28 | 2.24 | 86.58 |
| 30 C./2 M | Complies | Complies | Complies | 4.12 | 96.42 | 2.14 | 84.56 |
| 30 C./3 M | Complies | Complies | Complies | 4.18 | 95.07 | 2.17 | 81.99 |
| 25 C./1 M | Complies | Complies | Complies | 3.99 | 97.06 | 2.22 | 87.03 |
| 25 C./2 M | Complies | Complies | Complies | 4.08 | 96.40 | 2.14 | 88.87 |
| 25 C./3 M | Complies | Complies | Complies | 4.15 | 95.33 | 2.00 | 82.47 |

| | Analytical Data | | | |
|---|---|---|---|---|
| | Related Substances | | | |
| | | | Total | |
| | | Each | impurity | |
| | | individual | (Excluding | |
| | Impurity | unspecified | Imp F and D- | Particulate |
| Conditions | F | impurity | Epinephrine) | matter |
| specification | NMT | NMT | NMT | For ≥ 10 μm: |
| Limit | 7.5% | 0.2% | 10.0% | NMT 25 |
| | | | | particles |
| | | | | per mL |
| | | | | For ≥ 25 μm: |
| | | | | Not More Than |
| | | | | 3 particles |
| | | | | per mL |
| Initial | 2.68 | 0.03 | 0.03 | 10 μm: 1.5 |
| | | (RRT-1.08) | | 25 μm: 0.1 |
| 40 C./1 M | 4.19 | 0.022 | 0.040 | 10 μm: 01 |
| | | (RRT0.50) | | 25 μm: 00 |
| | | 0.018 | | |
| | | (RRT-1.09) | | |
| 40 C./2 M | 6.00 | 0.016 | 0.016 | 10 μm: 1.3 |
| | | (RRT-0.83) | | 25 μm: 0.1 |
| 40 C./3 M | 7.329 | 0.053 | 0.053 | NA |
| | | (RRT-0.51) | | |
| 30 C./1 M | 3.12 | 0.032 | 0.032 | 10 μm: 01 |
| | | (RRT-1.08) | | 25 μm: 00 |
| 30 C./2 M | 3.91 | 0.015 | 0.015 | 10 μm: 1.7 |
| | | (RRT-0.83) | | 25 μm: 0.1 |
| 30 C./3 M | 4.68 | ND | ND | NA |
| 25 C./1 M | 2.97 | 0.034 | 0.034 | 10 μm: 00 |
| | | (RRT-1.09) | | 25 μm: 00 |
| 25 C./2 M | 3.37 | ND | ND | 10 μm: 0.8 |
| | | | | 25 μm: 0.0 |
| 25 C./3 M | 4.223 | ND | ND | NA |

TABLE 4
Epinephrine 16 mcg/mL at pH 4.0

| | Analytical Data | | | | | | |
|---|---|---|---|---|---|---|---|
| | | Color and | | | Assay | | |
| | | achromicity | Clarity | | L- | D- | |
| Conditions | Description | of solution | of solution | pH | Epinephrine | Epinephrine | SMBS |
| specification | A clear and | Color of the | Clear solution | Between | 90.0 to | NMT | 10.0 to |
| Limit | colorless | solution | essentially | 3.0 and | 110.0% | 5.5% | 110.0% |
| | solution | should not be | free from | 5.0 | | | |
| | filled | more intense | visible | | | | |
| | in a bag | than Purified | particles | | | | |
| | | water. | | | | | |
| Initial | Complies | Complies | Complies | 4.16 | 96.39 | 1.76 | 71.48 |
| 40 C./1 M | Complies | Complies | Complies | 4.20 | 92.26 | 2.60 | 69.55 |
| 40 C./2 M | Complies | Complies | Complies | 4.20 | 92.43 | 1.64 | 44.4 |
| 40 C./3 M | Complies | Complies | Complies | 4.40 | 92.38 | 1.87 | 73.66 |
| 40 C./6 M | Complies | Complies | Complies | 4.42 | 87.75 | 2.31 | 74.33 |
| 25 C./1 M | Complies | Complies | Complies | 4.13 | 93.31 | 2.34 | 71.09 |
| 25 C./2 M | Complies | Complies | Complies | 4.05 | 94.93 | 1.40 | 46.28 |
| 25 C./3 M | Complies | Complies | Complies | 4.11 | 96.28 | 1.54 | 81.75 |
| 25 C./6 M | Complies | Complies | Complies | 4.24 | 94.92 | 1.69 | 83.05 |

| | Analytical Data | | | |
|---|---|---|---|---|
| | Related Substances | | | |
| | | | Total | |
| | | Each | impurity | |
| | | individual | (Excluding | |
| | Impurity | unspecified | Imp F and D- | Particulate |
| Conditions | F | impurity | Epinephrine) | matter |
| specification | NMT | NMT | NMT | For ≥ 10 μm: |
| Limit | 7.5% | 0.2% | 10.0% | NMT 25 |
| | | | | particles |
| | | | | per mL |
| | | | | For ≥ 25 μm: |
| | | | | Not More Than |
| | | | | 3 particles |
| | | | | per mL |
| Initial | 3.547 | 0.003(RRT-0.58) | 0.003 | NA |
| 40 C./1 M | 4.809 | ND | ND | NA |
| 40 C./2 M | 6.844 | 0.042(RRT 0.58) | 0.062 | NA |
| 40 C./3 M | 8.290 | 0.131(RRT-0.19) | 0.131 | NA |
| 40 C./6 M | 13.080 | ND | ND | 10 μm: 1.5 |
| | | | | 25 μm: 0.0 |
| 25 C./1 M | 3.507 | ND | ND | NA |
| 25 C./2 M | 3.911 | 0.038 (RRT 0.58) | 0.060 | NA |
| 25 C./3 M | 4.260 | 0.107(RRT-0.19) | 0.107 | NA |
| 25 C./6 M | 4.800 | ND | ND | 10 μm: 0.8 |
| | | | | 25 μm: 0.0 |

TABLE 5
Epinephrine 16 mcg/mL at pH 5.0

| | Analytical Data | | | | | | |
|---|---|---|---|---|---|---|---|
| | | Color and | | | Assay | | |
| | | achromicity | Clarity | | L- | D- | |
| Conditions | Description | of solution | of solution | pH | Epinephrine | Epinephrine | SMBS |
| specification | A clear and | Color of the | Clear solution | Between | 90.0 to | NMT | 10.0 to |
| Limit | colorless | solution | essentially | 3.0 and | 110.0% | 5.5% | 110.0% |
| | solution | should not be | free from | 5.0 | | | |
| | filled | more intense | visible | | | | |
| | in a bag | than Purified | particles | | | | |
| | | water. | | | | | |
| Initial | Complies | Complies | Complies | 4.95 | 92.39 | 1.18 | 89.9 |
| 40 C./1 M | Complies | Complies | Complies | 4.86 | 93.68 | 1.22 | 88.0 |
| 25 C./1 M | Complies | Complies | Complies | 5.01 | 94.79 | 1.05 | 92.8 |

| | Analytical Data | | | |
|---|---|---|---|---|
| | Related Substances | | | |
| | | | Total | |
| | | Each | impurity | |
| | | individual | (Excluding | |
| | Impurity | unspecified | Imp F and D- | Particulate |
| Conditions | F | impurity | Epinephrine) | matter |
| specification | NMT | NMT | NMT | For ≥ 10 μm: |
| Limit | 7.5% | 0.2% | 10.0% | NMT 25 |
| | | | | particles |
| | | | | per mL |
| | | | | For ≥ 25 μm: |
| | | | | Not More Than |
| | | | | 3 particles |
| | | | | per mL |
| Initial | 6.766 | 0.101(RRT-1.09) | 0.140 | 10 μm: 5.2 |
| | | 0.039(RRT-0.85) | | 25 μm: 0.1 |
| 40 C./1 M | 7.687 | 0.073(RRT-1.09) | 0.168 | NP |
| | | 0.046(RRT-0.85) | | |
| | | 0.037(RRT-0.95) | | |
| | | 0.012(RRT-0.27) | | |
| 25 C./1 M | 6.209 | 0.096(RRT-1.09) | 0.177 | NP |
| | | 0.037(RRT-0.95) | | |
| | | 0.032(RRT-0.85) | | |

TABLE 6
Epinephrine 16 mcg/mL at pH 6.0

| | Analytical Data | | | | | | |
|---|---|---|---|---|---|---|---|
| | | Color and | | | Assay | | |
| | | achromicity | Clarity | | L- | D- | |
| Conditions | Description | of solution | of solution | pH | Epinephrine | Epinephrine | SMBS |
| specification | A clear and | Color of the | Clear solution | Between | 90.0 to | NMT | 10.0 to |
| Limit | colorless | solution | essentially | 3.0 and | 110.0% | 5.5% | 110.0% |
| | solution | should not be | free from | 5.0 | | | |
| | filled | more intense | visible | | | | |
| | in a bag | than Purified | particles | | | | |
| | | water. | | | | | |
| Initial | Complies | Complies | Complies | 5.82 | 75.28 | 0.58 | 68.5 |
| 40 C./1 M | Complies | Complies | Complies | 5.72 | 72.22 | 0.50 | 55.4 |
| 40 C./2 M | Complies | Complies | Complies | 5.76 | 73.18 | 0.61 | 63.1 |
| 25 C./1 M | Complies | Complies | Complies | 5.77 | 73.6 | 0.49 | 64.7 |
| 25 C./2 M | Complies | Complies | Complies | 5.89 | 77.49 | 0.58 | 74.5 |

| | Analytical Data | | | |
|---|---|---|---|---|
| | Related Substances | | | |
| | | | Total | |
| | | Each | impurity | |
| | | individual | (Excluding | |
| | Impurity | unspecified | Imp F and D- | Particulate |
| Conditions | F | impurity | Epinephrine) | matter |
| specification | NMT | NMT | NMT | For ≥ 10 μm: |
| Limit | 7.5% | 0.2% | 10.0% | NMT 25 |
| | | | | particles |
| | | | | per mL |
| | | | | For ≥ 25 μm: |
| | | | | Not More Than |
| | | | | 3 particles |
| | | | | per mL |
| Initial | 13.947 | 0.223(RRT-1.09) | 0.287 | 10 μm: 6.9 |
| | | 0.064(RRT-0.84) | | 25 μm: 0.5 |
| 40 C./1 M | 9.405 | 0.448(RRT-1.09) | 0.863 | NP |
| | | 0.211(RRT-0.27) | | |
| | | 0.165(RRT-0.85) | | |
| | | 0.039(RRT-0.95) | | |
| 40 C./2 M | 13.446 | 0.179(RRT-0.84) | 0.833 | NP |
| | | 0.509(RRT-1.09) | | |
| | | 0.089(RRT-2.14) | | |
| 25 C./1 M | 11.816 | 0.232(RRT-1.09) | 0.686 | NP |
| | | 0.249(RRT-0.25) | | |
| | | 0.084(RRT-0.85) | | |
| | | 0.078(RRT-1.97) | | |
| 25 C./2 M | 9.159 | 0.213(RRT-1.09) | 0.549 | NP |
| | | 0.208(RRT-2.14) | | |
| | | 0.077(RRT-0.84) | | |

### Example 2

### Impact of Sodium Metabisulfite (SMBS) on Epinephrine Premix Formulations

The following example describes studies that tested the impact of sodium meta bisulfite (SMBS) on the stability of premix epinephrine formulations. The below tables provide stability data for liquid epinephrine formulations having varying concentrations of epinephrine (ranging from 16 mcg/mL to 64 mcg/mL) in 0.9% sodium chloride, with various concentrations of sodium meta bisulfite (SMBS; 0 to 0.1 mg/mL).

The data provided in Tables 7 to 20 shows that the addition of various concentrations of sodium metabisulfite (SMBS) as indicated stabilized the epinephrine premix formulation (more so than formulations without SMBS) as measured, for example, by the amount of D-epinephrine (impurity) in the formulation.

Tables 21 to 23 describe stability of epinephrine premix formulations having 0.032 mg/ml disodium edetate dihydrate (EDTA) and 0.05 mg/mL of SMBS.

TABLE 7
Epinephrine 32 mcg/mL with 0.1 mg/ml SMBS

| | | Analytical Data | | | | | | |
|---|---|---|---|---|---|---|---|---|
| | | | Color and | | | Assay | | |
| Trial | | | achromicity | Clarity | | L- | D- | |
| details | Conditions | Description | of solution | of solution | pH | Epinephrine | Epinephrine | SMBS |
| | A clear and | Color of the | Clear solution | Between | 90.0 to | NMT | 10.0 to | NMT |
| | colorless | solution | essentially | 3.0 and | 110.0% | 5.5% | 110.0% | 7.5% |
| | solution | should not be | free from | 5.0 | | | | |
| | filled | more intense | visible | | | | | |
| | in a bag | than Purified | particles | | | | | |
| | | water. | | | | | | |
| Trial | Initial | Complies | Complies | Complies | 4.05 | 98.70 | 1.11 | 79.5 |
| with | 40° C./1 M | Complies | Complies | Complies | 4.17 | 98.22 | 1.19 | 74.7 |
| SMBS | 40° C./2 M | Complies | Complies | Complies | 4.22 | 92.96 | 1.96 | 67.1 |
| (0.1 | 40° C./3 M | Complies | Complies | Complies | 4.42 | 91.35 | 2.06 | 61.1 |
| mg/mL) | 40° C./6 M | Complies | Complies | Complies | 4.40 | 94.68 | 1.53 | 62.4 |
| | 30° C./1 M | Complies | Complies | Complies | 4.15 | 99.55 | 1.12 | 76.4 |
| | 30° C./2 M | Complies | Complies | Complies | 4.24 | 94.04 | 1.82 | 65.3 |
| | 30° C./3 M | Complies | Complies | Complies | 4.26 | 94.29 | 1.67 | 68.5 |
| | 30° C./6 M | Complies | Complies | Complies | 4.34 | 98.88 | 1.21 | 70.4 |
| | 25° C./1 M | Complies | Complies | Complies | 4.10 | 99.14 | 1.11 | 74.6 |
| | 25° C./2 M | Complies | Complies | Complies | 4.25 | 94.61 | 1.66 | 63.2 |
| | 25° C./3 M | Complies | Complies | Complies | 4.36 | 94.88 | 1.73 | 72.1 |
| | 25° C./6 M | Complies | Complies | Complies | 4.31 | 100.26 | 1.07 | 71.8 |

| | | Analytical Data | | | |
|---|---|---|---|---|---|
| | | Related Substances | | | |
| | | | | Total | |
| | | | Each | impurity | |
| | | | individual | (Excluding | |
| Trial | | Impurity | unspecified | Imp F and D- | Particulate |
| details | Conditions | F | impurity | Epinephrine) | matter |
| | A clear and | NMT | NMT | NMT 6000 | A clear and |
| | colorless | 0.2% | 10.0% | per | colorless |
| | solution | | | container; | solution |
| | filled | | | NMT 600 | filled |
| | in a bag | | | per | in a bag |
| | | | | container | |
| Trial | Initial | 4.242 | ND | ND | NP |
| with | 40° C./1 M | 5.410 | ND | ND | NP |
| SMBS | 40° C./2 M | 7.548 | 0.042 (RRT-0.39) | 0.042 | NP |
| (0.1 | 40° C./3 M | 8.437 | 0.034 (RRT-0.4) | 0.099 | NP |
| mg/mL) | | | 0.065 (RRT-0.57) | | |
| | 40° C./6 M | 13.200 | 0.092 (RRT-0.71) | 0.132 | NP |
| | | | 0.040 (RRT-0.92) | | |
| | 30° C./1 M | 4.490 | ND | ND | NP |
| | 30° C./2 M | 5.313 | 0.048 (RRT-0.39) | 0.048 | NP |
| | 30° C./3 M | 5.436 | 0.037 (RRT-0.4) | 0.066 | NP |
| | | | 0.029 (RRT-0.57) | | |
| | 30° C./6 M | 6.960 | 0.081(RRT-0.72) | 0.118 | 10 μm: 473.3 |
| | | | 0.037(RRT-0.92) | | 25 μm: 20.0 |
| | 25° C./1 M | 4.320 | ND | ND | NP |
| | 25° C./2 M | 4.987 | 0.046 (RRT-0.4) | 0.046 | NP |
| | 25° C./3 M | 4.968 | 0.019 (RRT-0.4) | 0.019 | NP |
| | 25° C./6 M | 5.290 | ND | ND | 10 μm: 1180.0 |
| | | | | | 25 μm: 33.3 |

TABLE 8
Epinephrine 16 mcg/mL with 0.025 mg/ml SMBS

| | | Analytical Data | | | | | | |
|---|---|---|---|---|---|---|---|---|
| | | | Color and | | | Assay | | |
| Trial | | | achromicity | Clarity | | L- | D- | |
| details | Conditions | Description | of solution | of solution | pH | Epinephrine | Epinephrine | SMBS |
| | | A clear and | Color of the | Clear solution | Between | 90.0 to | NMT | 10.0 to |
| | | colorless | solution | essentially | 3.0 and | 110.0% | 5.5% | 110.0% |
| | | solution | should not be | free from | 5.0 | | | |
| | | filled | more intense | visible | | | | |
| | | in a bag | than Purified | particles | | | | |
| | | | water. | | | | | |
| Trial | Initial | Complies | Complies | Complies | 3.92 | 97.0 | 1.87 | 53.7 |
| with | | | | | | | | |
| SMBS | 1 M/25 C. | Complies | Complies | Complies | 3.86 | 99.7 | 2.14 | 71.26 |
| (0.025 | | | | | | | | |
| mg/mL) | | | | | | | | |

| | | Analytical Data | | | |
|---|---|---|---|---|---|
| | | Related Substances | | | |
| | | | | Total | |
| | | | Each | impurity | |
| | | | individual | (Excluding | |
| Trial | | Impurity | unspecified | Imp F and D- | Particulate |
| details | Conditions | F | impurity | Epinephrine) | matter |
| | | NMT | NMT | NMT | NMT 6000 |
| | | 7.5% | 0.2% | 10.0% | per |
| | | | | | container |
| | | | | | NMT 600 |
| | | | | | per |
| | | | | | container |
| Trial | Initial | 1.455 | 0.121(RRT-0.85) | 0.145 | NP |
| with | | | 0.024(RRT-0.58) | | |
| SMBS | 1 M/25 C. | 1.54 | 0.114(RRT-0.92) | 0.177 | NP |
| (0.025 | | | 0.063(RRT-0.16) | | |
| mg/mL) | | | | | |

NOTE:
Conditions are described in terms of temperature and time periods. For example, “1 M/25 C.” indicates the formulation was kept at 25 degrees Celsius for 1 month and then tested. “40 C./1 M” indicates the formulation was kept at 40 degrees Celsius for 1 month and then tested.

TABLE 9
Epinephrine 16 mcg/mL with 0.05 mg/ml SMBS

| | | Analytical Data | | | | | | |
|---|---|---|---|---|---|---|---|---|
| | | | Color and | | | Assay | | |
| Trial | | | achromicity of | Clarity | | L- | D- | |
| details | Conditions | Description | solution | of solution | pH | Epinephrine | Epinephrine | SMBS |
| | | A clear and | Color of the | Clear solution | Between | 90.0 to | NMT | 10.0 to |
| | | colorless | solution | essentially | 3.0 and | 110.0% | 5.5% | 110.0 |
| | | solution | should not be | free from | 5.0 | | | % |
| | | filled | more intense | visible | | | | |
| | | in a bag | than Purified | particles | | | | |
| | | | water. | | | | | |
| Trial | Initial | Complies | Complies | Complies | 4.14 | 98.22 | 1.77 | 87.75 |
| with | 40 C./1 M | Complies | Complies | Complies | 4.07 | 96.47 | 2.40 | 83.32 |
| SMBS | 40 C./2 M | Complies | Complies | Complies | 4.12 | 94.47 | 2.45 | 82.88 |
| (0.05 | 40 C./3 M | Complies | Complies | Complies | 4.23 | 92.12 | 2.51 | 71.30 |
| mg/mL) | 30 C./1 M | Complies | Complies | Complies | 3.98 | 97.28 | 2.24 | 86.58 |
| | 30 C./2 M | Complies | Complies | Complies | 4.12 | 96.42 | 2.14 | 84.56 |
| | 30 C./3 M | Complies | Complies | Complies | 4.18 | 95.07 | 2.17 | 81.99 |
| | 25 C./1 M | Complies | Complies | Complies | 3.99 | 97.06 | 2.22 | 87.03 |
| | 25 C./2 M | Complies | Complies | Complies | 4.08 | 96.40 | 2.14 | 88.87 |
| | 25 C./3 M | Complies | Complies | Complies | 4.15 | 95.33 | 2.00 | 82.47 |

| | | Analytical Data | | | |
|---|---|---|---|---|---|
| | | Related Substances | | | |
| | | | | Total | |
| | | | Each | impurity | |
| | | | individual | (Excluding | |
| Trial | | Impurity | unspecified | Imp F and D- | Particulate |
| details | Conditions | F | impurity | Epinephrine) | matter |
| | | NMT | NMT | NMT | For ≥ 10 μm: |
| | | 7.5% | 0.2% | 10.0% | NMT 25 |
| | | | | | particles |
| | | | | | per mL |
| | | | | | For ≥ 25 μm: |
| | | | | | Not More Than |
| | | | | | 3 particles |
| | | | | | per mL |
| Trial | Initial | 2.68 | 0.03 | 0.03 | 10 μm: 1.5 |
| with | | | (RRT-1.08) | | 25 μm: 0.1 |
| SMBS | 40 C./1 M | 4.19 | 0.022 | 0.04 | 10 μm: 01 |
| (0.05 | | | (RRT0.50) | | 25 μm: 00 |
| mg/mL) | | | 0.018 | | |
| | | | (RRT-1.09) | | |
| | 40 C./2 M | 6.00 | 0.016 | 0.016 | 10 μm: 1.3 |
| | | | (RRT-0.83) | | 25 μm: 0.1 |
| | 40 C./3 M | 7.33 | 0.053 | 0.053 | NA |
| | | | (RRT-0.51) | | |
| | 30 C./1 M | 3.12 | 0.032 | 0.032 | 10 μm: 01 |
| | | | (RRT-1.08) | | 25 μm: 00 |
| | 30 C./2 M | 3.91 | 0.015 | 0.015 | 10 μm: 1.7 |
| | | | (RRT-0.83) | | 25 μm: 0.1 |
| | 30 C./3 M | 4.68 | ND | ND | NA |
| | 25 C./1 M | 2.97 | 0.034 | 0.034 | 10 μm: 00 |
| | | | (RRT-1.09) | | 25 μm: 00 |
| | 25 C./2 M | 3.37 | ND | ND | 10 μm: 0.8 |
| | | | | | 25 μm: 0.0 |
| | 25 C./3 M | 4.22 | ND | ND | NA |

TABLE 10
Epinephrine 16 mcg/mL with 0.1 mg/ml SMBS

| | | Analytical Data | | | | | | |
|---|---|---|---|---|---|---|---|---|
| | | | Color and | | | Assay | | |
| Trial | | | achromicity | Clarity | | L- | D- | |
| details | Conditions | Description | of solution | of solution | pH | Epinephrine | Epinephrine | SMBS |
| | | A clear and | Color of the | Clear solution | Between | 90.0 to | NMT | 10.0 to |
| | | colorless | solution | essentially | 3.0 and | 110.0% | 5.5% | 110.0% |
| | | solution | should not be | free from | 5.0 | | | |
| | | filled | more intense | visible | | | | |
| | | in a bag | than Purified | particles | | | | |
| | | | water. | | | | | |
| | Initial | Complies | Complies | Complies | 4.00 | 97.64 | 1.05 | 81.69 |
| Trial | 40° C./1 M | Complies | Complies | Complies | 4.34 | 94.79 | 1.30 | 76.36 |
| with | 40° C./2 M | Complies | Complies | Complies | 4.28 | 91.62 | 1.76 | 73.78 |
| SMBS | 40° C./3 M | Complies | Complies | Complies | 4.29 | 90.53 | 0.99 | 61.46 |
| (0.1 | 30° C./1 M | Complies | Complies | Complies | 4.33 | 95.68 | 1.20 | 76.95 |
| mg/mL) | 30° C./2 M | Complies | Complies | Complies | 4.21 | 93.03 | 1.72 | 76.70 |
| | 30° C./3 M | Complies | Complies | Complies | 4.20 | 93.60 | 0.78 | 62.34 |
| | 25° C./1 M | Complies | Complies | Complies | 4.32 | 96.92 | 0.80 | 79.60 |
| | 25° C./2 M | Complies | Complies | Complies | 4.16 | 93.37 | 1.74 | 78.66 |
| | 25° C./3 M | Complies | Complies | Complies | 4.21 | 94.28 | 0.90 | 66.13 |
| | 25° C./6 M | Complies | Complies | Complies | 4.28 | 92.75 | 1.02 | 90.96 |

| | | Analytical Data | | | |
|---|---|---|---|---|---|
| | | Related Substances | | | |
| | | | | Total | |
| | | | Each | impurity | |
| | | | individual | (Excluding | |
| Trial | | Impurity | unspecified | Imp F and D- | Particulate |
| details | Conditions | F | impurity | Epinephrine) | matter |
| | | NMT | NMT | NMT | For ≥ 10 μm: |
| | | 7.5% | 0.2% | 10.0% | NMT 25 |
| | | | | | particles |
| | | | | | per mL |
| | | | | | For ≥ 25 μm: |
| | | | | | Not More Than |
| | | | | | 3 particles |
| | | | | | per mL |
| | Initial | 4.63 | 0.055 | 0.055 | NA |
| | | | (RRT-0.29) | | |
| Trial | 4O° C./1 M | 6.62 | 0.041 | 0.041 | NA |
| with | | | (RRT-0.58) | | |
| SMBS | 40° C./2 M | 8.04 | ND | 0.012 | NA |
| (0.1 | 40° C./3 M | 10.18 | ND | ND | NA |
| mg/mL) | 30° C./1 M | 5.62 | 0.015 | 0.015 | NA |
| | | | (RRT-0.58) | | |
| | 30° C./2 M | 5.76 | ND | ND | NA |
| | 30° C./3 M | 6.15 | ND | ND | NA |
| | 25° C./1 M | 5.44 | 0.008 | 0.008 | NA |
| | | | (RRT-0.58) | | |
| | 25° C./2 M | 5.27 | ND | ND | NA |
| | 25° C./3 M | 5.61 | 0.015 | 0.015 | NA |
| | | | (RRT-0.32) | | |
| | 25° C./6 M | 6.76 | 0.058 | 0.058 | 10 μm: 0.9 |
| | | | (RRT-0.54) | | 25 μm: 0.1 |

TABLE 11
Epinephrine 32 mcg/mL with 0.025 mg/ml SMBS

| | | Analytical Data | | | | | | |
|---|---|---|---|---|---|---|---|---|
| | | | Color and | | | | | |
| | | | achromicity | | | | | |
| | | | of solution | Clarity | | | | |
| | | | Color of the | of solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial with | Initial | Complies | Complies | Complies | 3.86 | 97.37 | 2.27 | 53.62 |
| SMBS | 1M/25 C. | Complies | Complies | Complies | 3.91 | 99.8 | 2.27 | 58.9 |
| (0.025 | | | | | | | | |
| mg/mL) | | | | | | | | |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial with | 1.05 | 0.123(RRT-0.85) | 0.164 | NP |
| SMBS | | 0.026(RRT-0.58) | | |
| (0.025 | 1.51 | 0.407(RRT-1.03) | 0.67 | NP |
| mg/mL) | | 0.140(RRT-0.92) | | |
| | | 0.088(RRT-0.38) | | |
| | | 0.035(RRT-0.21) | | |

TABLE 12
Epinephrine 32 mcg/mL with 0.05 mg/ml SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial with | Initial | Complies | Complies | Complies | 3.88 | 98.02 | 2.28 | 82.60 |
| SMBS | 40 C./1M | Complies | Complies | Complies | 3.76 | 96.58 | 2.96 | 75.35 |
| (0.05 | 40 C./2M | Complies | Complies | Complies | 3.89 | 94.99 | 4.21 | 71.19 |
| mg/mL) | 40 C./3M | Complies | Complies | Complies | 3.98 | 93.87 | 3.59 | 67.52 |
| | 30 C./1M | Complies | Complies | Complies | 3.72 | 97.44 | 2.63 | 81.25 |
| | 30 C./2M | Complies | Complies | Complies | 3.83 | 95.95 | 3.36 | 76.69 |
| | 30 C./3M | Complies | Complies | Complies | 3.86 | 96.03 | 2.73 | 71.35 |
| | 25 C./1M | Complies | Complies | Complies | 3.7 | 97.85 | 2.56 | 83.12 |
| | 25 C./2M | Complies | Complies | Complies | 3.77 | 95.23 | 3.13 | 69.66 |
| | 25 C./3M | Complies | Complies | Complies | 3.83 | 97.44 | 2.56 | 77.07 |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial with | 1.76 | ND | ND | 10 μm: 1.3 |
| SMBS | | | | 25 μm: 0.1 |
| (0.05 | 2.95 | ND | ND | 10 μm: 0 |
| mg/mL) | | | | 25 μm: 0 |
| | 4.16 | ND | ND | 10 μm: 0.9 |
| | | | | 25 μm: 0.1 |
| | 6.24 | ND | ND | NA |
| | 2.16 | ND | ND | 10 μm: 0 |
| | | | | 25 μm: 0 |
| | 2.48 | ND | ND | 10 μm: 2.9 |
| | | | | 25 μm: 0.2 |
| | 3.80 | ND | ND | NA |
| | 1.94 | ND | ND | 10 μm: 0 |
| | | | | 25 μm: 0 |
| | 2.55 | 0.045 (RRT-1.08) | ND | 10 μm: 1.1 |
| | | 0.026 (RRT-0.83) | | 25 μm: 0.1 |
| | | 0.023 (RRT-0.52) | | |
| | 3.261 | ND | ND | NA |

TABLE 13
Epinephrine 32 mcg/mL with 0.1 mg/ml SMBS

| | | Analytical Data | | | | | | |
|---|---|---|---|---|---|---|---|---|
| | | | Color and | | | | | |
| | | Description | achromicity | | | | | |
| | | Color of the | of solution | | | Assay | | |
| | Conditions | solution should | Clear solution | Clarity of | | | D- | |
| | A clear and | not be more | essentially | solution | pH | L- | Epinephrine | |
| Trial | colorless solution | intense than | free from visible | Between | 90.0 to | Epinephrine | 10.0 to | SMBS |
| details | filled in a bag | Purified water. | particles | 3.0 and 5.0 | 110.0% | NMT 5.5% | 110.0% | NMT 7.5% |
| Trial with | Initial | Complies | Complies | Complies | 4.05 | 98.70 | 1.11 | 79.49 |
| SMBS | 40° C./1M | Complies | Complies | Complies | 4.17 | 98.22 | 1.19 | 74.74 |
| (0.1 | 40° C./2M | Complies | Complies | Complies | 4.22 | 92.96 | 1.96 | 67.07 |
| mg/mL) | 40° C./3M | Complies | Complies | Complies | 4.42 | 91.35 | 2.06 | 61.13 |
| | 40° C./6M | Complies | Complies | Complies | 4.40 | 94.68 | 1.53 | 62.35 |
| | 25° C./1M | Complies | Complies | Complies | 4.10 | 99.14 | 1.11 | 74.63 |
| | 25° C./2M | Complies | Complies | Complies | 4.25 | 94.61 | 1.66 | 63.18 |
| | 25° C./3M | Complies | Complies | Complies | 4.36 | 94.88 | 1.73 | 72.08 |
| | 25° C./6M | Complies | Complies | Complies | 4.31 | 100.26 | 1.07 | 71.84 |

| | Analytical Data | | | |
|---|---|---|---|---|
| | Related Substances | | | |
| | | | Total impurity | |
| | | | (Excluding Imp F and D- | |
| | | | Epinephrine) | |
| | | | For ≥10 μm: NMT | |
| | | | 25 particles per mL | Particulate matter |
| | | Each individual | For ≥25 μm: | A clear and |
| Trial | Impurity F | unspecified impurity | Not More Than | colorless solution |
| details | NMT 0.2% | NMT 10.0% | 3 particles per mL | filled in a bag |
| Trial with | 4.24 | ND | ND | NP |
| SMBS | 5.41 | ND | ND | NP |
| (0.1 | 7.55 | 0.042 (RRT-0.39) | 0.042 | NP |
| mg/mL) | 8.44 | 0.034 (RRT-0.4) | 0.099 | NP |
| | | 0.065 (RRT-0.57) | | |
| | 13.20 | 0.092 (RRT-0.71) | 0.132 | NP |
| | | 0.040 (RRT-0.92) | | |
| | 4.32 | ND | ND | NP |
| | 4.99 | 0.046 (RRT-0.4) | 0.046 | NP |
| | 4.97 | 0.019 (RRT-0.4) | 0.019 | NP |
| | 5.29 | ND | ND | 10 μm: 1180.0 |
| | | | | 25 μm: 33.3 |

TABLE 14
Epinephrine 64 mcg/mL epinephrine with 0.025 mg/ml SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial with | Initial | Complies | Complies | Complies | 3.95 | 97.37 | 2.26 | 54.1 |
| SMBS | 1M/25 C. | Complies | Complies | Complies | 3.91 | 98.23 | 1.60 | 61.0 |
| (0.025 | | | | | | | | |
| mg/mL) | | | | | | | | |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial with | 1.06 | 0.026(RRT-0.85) | 0.049 | NP |
| SMBS | 1.45 | 0.093(RRT-0.22) | 0.093 | NP |
| (0.025 | | | | |
| mg/mL) | | | | |

TABLE 15
Epinephrine 64 mcg/mL with 0.05 mg/ml SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial with | Initial | Complies | Complies | Complies | 3.91 | 99.80 | 2.53 | 83.4 |
| SMBS | 40 C./1M | Complies | Complies | Complies | 3.78 | 95.34 | 2.77 | 76.0 |
| (0.05 | 40 C./2M | Complies | Complies | Complies | 3.93 | 95.56 | 3.66 | NA |
| mg/mL) | 40 C./3M | Complies | Complies | Complies | 3.96 | 93.68 | 3.38 | 65.3 |
| | 30 C./1M | Complies | Complies | Complies | 3.75 | 95.64 | 2.44 | 81.3 |
| | 30 C./2M | Complies | Complies | Complies | 3.83 | 96.91 | 3.02 | 76.41 |
| | 30 C./3M | Complies | Complies | Complies | 3.91 | 96.84 | 2.53 | 71.5 |
| | 25 C./1M | Complies | Complies | Complies | 3.72 | 96.86 | 2.35 | 81.8 |
| | 25 C./2M | Complies | Complies | Complies | 3.86 | 97.01 | 2.70 | 79.6 |
| | 25 C./3M | Complies | Complies | Complies | 3.87 | 96.71 | 2.45 | 76.5 |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial with | 2.02 | ND | ND | 10 μm: 1.3 |
| SMBS | | | | 25 μm: 0.0 |
| (0.05 | 3.16 | ND | ND | 10 μm: 01 |
| mg/mL) | | | | 25 μm: 0.0 |
| | 4.43 | ND | ND | 10 μm: 2.0 |
| | | | | 25 μm: 0.1 |
| | 5.95 | ND | ND | NA |
| | 2.25 | ND | ND | 10 μm: 03 |
| | | | | 25 μm: 0.0 |
| | 2.64 | ND | ND | 10 μm: 0.6 |
| | | | | 25 μm: 0.1 |
| | 3.66 | ND | ND | NA |
| | 2.05 | ND | ND | 10 μm: 04 |
| | | | | 25 μm: 0.0 |
| | 2.25 | ND | ND | 10 μm: 0.8 |
| | | | | 25 μm: 0.1 |
| | 3.24 | ND | ND | NA |

TABLE 16
Epinephrine 64 mcg/mL with 0.1 mg/ml SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial with | Initial | Complies | Complies | Complies | 4.06 | 99.32 | 1.05 | 83.4 |
| SMBS | 40° C./1M | Complies | Complies | Complies | 4.30 | 95.46 | 1.13 | 78.7 |
| (0.1 | 40° C./2M | Complies | Complies | Complies | 4.18 | 95.71 | 0.96 | 76.2 |
| mg/mL) | 40° C./3M | Complies | Complies | Complies | 4.34 | 92.99 | 1.36 | 56.0 |
| | 40° C./6M | Complies | Complies | Complies | 4.28 | 86.07 | 1.27 | 77.9 |
| | 25° C./1M | Complies | Complies | Complies | 4.29 | 96.44 | 1.07 | 82.1 |
| | 25° C./2M | Complies | Complies | Complies | 4.16 | 97.90 | 0.86 | 81.3 |
| | 25° C./3M | Complies | Complies | Complies | 4.30 | 96.36 | 1.26 | 63.9 |
| | 25° C./6M | Complies | Complies | Complies | 4.24 | 93.40 | 0.87 | 90.7 |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial with | 4.42 | ND | ND | NP |
| SMBS | 6.19 | 0.040(RRT-0.59) | 0.062 | NP |
| (0.1 | 7.85 | ND | ND | NP |
| mg/mL) | 9.15 | 0.077(RRT-0.59) | 0.077 | NP |
| | 15.35 | 0.098(RRT-0.54) | 0.098 | 10 μm: 2.0 |
| | | | | 25 μm: 0.3 |
| | 5.06 | 0.036(RRT-0.59) | 0.061 | NP |
| | 5.03 | ND | ND | NP |
| | 5.05 | 0.043(RRT-0.58) | 0.043 | NP |
| | 6.21 | ND | ND | 10 μm: 3.8 |
| | | | | 25 μm: 0.0 |

TABLE 17
Epinephrine 16 mcg/mL without SMBS

| | | Analytical Data | | | | | | |
|---|---|---|---|---|---|---|---|---|
| | | | Color and | | | | | |
| | | | achromicity | | | | | |
| | | | of solution | Clarity | | | | |
| | | | Color of the | of solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial | Initial | Complies | Complies | Complies | 4.01 | 98.13 | 1.07 | NA |
| without | 40° C./1M | Complies | Complies | Complies | 4.05 | 96.71 | 2.64 | NA |
| SMBS | | | | | | | | |
| | 40° C./2M | Complies | Complies | Complies | 3.95 | 96.77 | 3.5 | NA |
| | 40° C./3M | Complies | Complies | Complies | 3.97 | 95.84 | 4.34 | NA |
| | 40° C./6M | Complies | Complies | Complies | 3.94 | 92.01 | 7.07 | NA |
| | 25° C./1M | Complies | Complies | Complies | 4.04 | 98.15 | 2.26 | NA |
| | 25° C./2M | Complies | Complies | Complies | 3.94 | 98.79 | 2.50 | NA |
| | 25° C./3M | Complies | Complies | Complies | 3.95 | 100.43 | 2.56 | NA |
| | 25° C./6M | Complies | Complies | Complies | 3.92 | 98.04 | 3.46 | NA |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial | ND | 0.256 (RRT-0.84) | 0.256 | NP |
| without | ND | 0.369 (RRT-0.84) | 0.639 | NP |
| SMBS | | 0.156 (RRT 0.09) | | |
| | ND | 1.115(RRT-1.04) | 2.976 | NP |
| | ND | 0.539(RRT-1.07) | 0.972 | 10 μm: 13.1 |
| | | 0.344(RRT-0.94) | | 25 μm: 0.5 |
| | | 0.089(RRT-0.74) | | |
| | ND | 0.170(RRT-1.67) | 2.726 | 10 μm: 01 |
| | | 0.693(RRT-0.83) | | 25 μm: 00 |
| | | 0.889(RRT-0.08) | | |
| | | 0.772(RRT-1.09) | | |
| | ND | 0.285(RRT-0.85) | 0.316 | NP |
| | ND | 1.042(RRT-1.04) | 2.099 | NP |
| | ND | 0.806(RRT-0194) | 2.914 | NP |
| | | 0.791(RRT-1.07) | | |
| | | 0.478(RRT-0.19) | | |
| | | 0.379(RRT-0.18) | | |
| | | 0.128(RRT-1.89) | | |
| | ND | 0.523(RRT-1.09) | 1.268 | 10 μm: 01 |
| | | 0.326(RRT-0.83) | | 25 μm: 00 |
| | | 0.144(RRT-0.08) | | |
| | | 0.167(RRT-1.73) | | |

TABLE 18
Epinephrine 32 mcg/mL without SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial | Initial | Complies | Complies | Complies | 4.24 | 93.41 | 2.44 | NA |
| without | 40° C./1M | Complies | Complies | Complies | 4.08 | 98.57 | 2.49 | NA |
| SMBS | 40° C./2M | Complies | Complies | Complies | 4.08 | 97.75 | 3.21 | NA |
| | 40° C./3M | Complies | Complies | Complies | 4.16 | 96.02 | 4.15 | NA |
| | 40° C./6M | Complies | Complies | Complies | 4.19 | 93.09 | 5.76 | NA |
| | 25° C./1M | Complies | Complies | Complies | 4.08 | 99.64 | 2.13 | NA |
| | 25° C./2M | Complies | Complies | Complies | 4.07 | 100.13 | 2.06 | NA |
| | 25° C./3M | Complies | Complies | Complies | 4.15 | 98.65 | 2.52 | NA |
| | 25° C./6M | Complies | Complies | Complies | 4.35 | 98.01 | 2.95 | NA |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial | ND | 0.160(RRT-0.84) | 0.160 | NP |
| without | ND | 0.216(RRT-0.85) | 0.529 | NP |
| SMBS | | 0.210(RRT-0.09) | | |
| | | 0.045(RRT-1.32) | | |
| | ND | 0.510(RRT-0.09) | 1.149 | NP |
| | | 0.317(RRT-0.85) | | |
| | | 0.140(RRT-1.68) | | |
| | | 0.050(RRT-0.78) | | |
| | ND | 0.636(RRT-1.04) | 2.633 | 10 μm: 28.9 |
| | | 0.558(RRT-0.17) | | 25 μm: 1.3 |
| | | 0.466(RRT-0.2) | | |
| | | 0.287(RRT-0.16) | | |
| | | 0.397(RRT-0.92) | | |
| | | 0.112(RRT-1.17) | | |
| | | 0.097(RRT-1.08) | | |
| | ND | 1.605(RRRT-0.08) | 3.318 | NP |
| | | 0.746(RRT-1.07) | | |
| | | 0.529(RRT-0.83) | | |
| | | 0.243(RRT-1.62) | | |
| | | 0.116RRT-0.51) | | |
| | | 0.079(RRT-0.70) | | |
| | ND | 0.167(RRT-0.85) | 0.213 | NP |
| | | 0.025(RRT-1.14) | | |
| | ND | 0.174(RRT-0.85) | 0.197 | NP |
| | ND | 0.543(RRT-1.04) | 2.092 | 10 μm: 13.4 |
| | | 0.512(RRT-0.2) | | 25 μm: 0.4 |
| | | 0.310(RRT-0.92) | | |
| | | 0.238(RRT-0.17) | | |
| | | 0.099(RRT-1.17) | | |
| | | 0.117(RRT-1.16) | | |
| | ND | 0.457(RRT-1.03) | 1.285 | NP |
| | | 0.405(RRT-0.08) | | |
| | | 0.271(RRT-0.83) | | |
| | | 0.101(RRT-0.70) | | |
| | | 0.051(RRT-0.51) | | |

TABLE 19
Epinephrine 64 mcg/mL without SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial | Initial | Complies | Complies | Complies | 3.83 | 95.29 | 2.25 | NA |
| without | 40° C./1M | Complies | Complies | Complies | 4.06 | 94.52 | 3.30 | NA |
| SMBS | 40° C./2M | Complies | Complies | Complies | 3.95 | 93.05 | 3.71 | NA |
| | 40° C./3M | Complies | Complies | Complies | 3.94 | 91.43 | 4.30 | NA |
| | 40° C./6M | Complies | Complies | Complies | 3.95 | 90.85 | 6.59 | NA |
| | 30° C./1M | Complies | Complies | Complies | 4.06 | 95.37 | 2.84 | NA |
| | 30° C./2M | Complies | Complies | Complies | 3.94 | 92.73 | 2.87 | NA |
| | 30° C./3M | Complies | Complies | Complies | 3.93 | 93.81 | 2.95 | NA |
| | 30° C./6M | Complies | Complies | Complies | 3.94 | 95.07 | 3.61 | NA |
| | 25° C./1M | Complies | Complies | Complies | 4.06 | 95.17 | 2.74 | NA |
| | 25° C./2M | Complies | Complies | Complies | 3.92 | 94.61 | 2.74 | NA |
| | 25° C./3M | Complies | Complies | Complies | 3.93 | 93.58 | 2.77 | NA |
| | 25° C./6M | Complies | Complies | Complies | 3.94 | 95.05 | 3.01 | NA |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial | ND | ND | ND | NP |
| without | ND | 0.072 (RRT-0.84) | 0.072 | NP |
| SMBS | ND | 0.242(RRT-0.09) | 0.478 | NP |
| | | 0.099(RRT-0.85) | | |
| | | 0.043(RRT-1.31) | | |
| | ND | 0.325(RRT-0.09) | 0.580 | 10 μm: 12.7 |
| | | 0.110(RRT-0.85) | | 25 μm: 0.8 |
| | | 0.045(RRT-0.58) | | |
| | ND | 0.125(RRT-2.21) | 1.488 | 10 μm: 4.8 |
| | | 0.087(RRT-1.77) | | 25 μm: 0.1 |
| | | 0.574(RRT-0.09) | | |
| | | 0.147(RRT-0.84) | | |
| | | 0.117(RRT-1.85) | | |
| | | 0.092(RRT-1.08) | | |
| | ND | 0.057(RRT-0.84) | 0.057 | NP |
| | ND | 0.107(RRT-0.09) | 0.257 | NP |
| | | 0.064(RRT-0.85) | | |
| | ND | 0.118(RRT-0.09) | 0.291 | 10 μm: 56.7 |
| | | 0.074(RRT-0.85) | | 25 μm: 1.5 |
| | ND | 0.075(RRT-2.21) | 0.679 | 10 μm: 1.9 |
| | | 0.284(RRT-0.09) | | 25 μm: 0.0 |
| | | 0.107(RRT-0.84) | | |
| | | 0.067(RRT-1.08) | | |
| | ND | 0.061(RRT-0.84) | 0.061 | NP |
| | ND | 0.054(RRT-0.85) | 0.164 | NP |
| | ND | 0.052(RRT-0.85) | 0.156 | NP |
| | | 0.048(RRT-0.09) | | |
| | ND | 0.069(RRT-2.21) | 0.744 | 10 μm: 3.1 |
| | | 0.053(RRT-2.17) | | 25 μm: 0.2 |
| | | 0.296(RRT-0.09) | | |
| | | 0.087(RRT-084) | | |

TABLE 20
Epinephrine 64 mcg/mL with 0.1 mg/ml SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial with | Initial | Complies | Complies | Complies | 4.27 | 98.80 | 0.75 | 84.55 |
| SMBS | 40° C./1M | Complies | Complies | Complies | 4.29 | 97.34 | 0.84 | 75.80 |
| (0.1 | 40° C./2M | Complies | Complies | Complies | 4.45 | 92.33 | 1.41 | 66.73 |
| mg/mL) | 40° C./3M | Complies | Complies | Complies | 4.58 | 91.05 | 1.45 | 63.57 |
| | 40° C./6M | Complies | Complies | Complies | 4.40 | 91.84 | 0.84 | 52.08 |
| | 30° C./1M | Complies | Complies | Complies | 4.29 | 99.17 | 0.80 | 74.19 |
| | 30° C./2M | Complies | Complies | Complies | 4.40 | 94.45 | 1.36 | 70.93 |
| | 30° C./3M | Complies | Complies | Complies | 4.50 | 93.44 | 1.42 | 68.95 |
| | 30° C./6M | Complies | Complies | Complies | 4.53 | 98.08 | 0.58 | 75.13 |
| | 25° C./1M | Complies | Complies | Complies | 4.33 | 97.42 | 0.87 | 77.29 |
| | 25° C./2M | Complies | Complies | Complies | 4.35 | 94.11 | 1.33 | 70.93 |
| | 25° C./3M | Complies | Complies | Complies | 4.35 | 92.52 | 1.46 | 69.38 |
| | 25° C./6M | Complies | Complies | Complies | 4.47 | 99.03 | 0.57 | 73.66 |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial with | 5.658 | ND | ND | NP |
| SMBS | 6.610 | ND | ND | NP |
| (0.1 | 8.603 | 0.056 (RRT-0.39) | 0.056 | NP |
| mg/mL) | 9.916 | 0.058 (RRT-0.39) | 0.106 | NP |
| | | 0.048 (RRT-0.57) | | |
| | 13.950 | 0.189(RRT-0.72) | 0.270 | 10 μm: 1633.3 |
| | | 0.049(RRT-0.40) | | 25 μm: 46.7 |
| | | 0.032(RRT-0.93) | | |
| | 5.460 | ND | ND | NP |
| | 6.157 | 0.057 (RRT-0.39) | 0.057 | NP |
| | 6.345 | 0.052 (RRT-0.39) | 0.052 | NP |
| | 7.880 | 0.127(RRT-0.72) | 0.204 | 10 μm: 693.3 |
| | | 0.048(RRT-0.40) | | 25 μm: 33.3 |
| | | 0.029(RRT-0.92) | | |
| | 6.750 | ND | ND | NP |
| | 5.623 | 0.046 (RRT-0.39) | 0.046 | NP |
| | 7.440 | 0.059 (RRT-0.39) | 0.087 | NP |
| | | 0.028 (RRT-0.58) | | |
| | 7.010 | 0.051(RRT-0.40) | 0.082 | 10 μm: 3160.0 |
| | | 0.031(RRT-0.93) | | 25 μm: 153.3 |

TABLE 21
Epinephrine 16 mcg/mL with 0.032 mg/ml edetate disodium dihydrate (EDTA) and 0.05 mg/mL of SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial | Initial | Complies | Complies | Complies | 4.04 | 96.6 | 2.91 | 97.30 |
| with | 40° C./1M | Complies | Complies | Complies | 4.04 | 96.5 | 2.78 | 84.10 |
| Disodium | 40° C./2M | Complies | Complies | Complies | 4.21 | 93.02 | 3.45 | 83.60 |
| EDTA | 25° C./1M | Complies | Complies | Complies | 3.98 | 97.72 | 2.85 | 90.40 |
| (0.032 | 25° C./2M | Complies | Complies | Complies | 4.16 | 95.64 | 3.02 | 90.9 |
| mg/mL) | | | | | | | | |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial | 2.122 | ND | ND | 10 μm: 07 |
| with | | | | 25 μm: 00 |
| Disodium | 3.675 | 0.020(RRT-0.51) | 0.052 | 10 μm: 03 |
| EDTA | | 0.017(RRT-0.57) | | 25 μm: 01 |
| (0.032 | | 0.015(RRT-0.71) | | |
| mg/mL) | 5.197 | ND | ND | 10 μm: 02 |
| | | | | 25 μm: 00 |
| | 2.354 | 0.023(RRT-1.09) | 0.076 | 10 μm: 02 |
| | | | | 25 μm: 00 |
| | | 0.020(RRT-0.57) | | |
| | | 0.017(RRT-0.51) | | |
| | 2.561 | ND | ND | 10 μm: 08 |
| | | | | 25 μm: 00 |

TABLE 22
Epinephrine 32 mcg/mL with 0.032 mg/ml edetate disodium dihydrate (EDTA) and 0.05 mg/mL of SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | | |
| | | A clear and | not be more | essentially | pH | Epinephrine | D- | SMBS |
| Trial | | colorless solution | intense than | free from visible | Between | 90.0 to | Epinephrine | 10.0 to |
| details | Conditions | filled in a bag | Purified water. | particles | 3.0 and 5.0 | 110.0% | NMT 5.5% | 110.0% |
| Trial | Initial | Complies | Complies | Complies | 4.1 | 96.82 | 2.97 | 89.1 |
| with | 40° C./1M | Complies | Complies | Complies | 4.10 | 96.14 | 3.30 | 81.30 |
| Disodium | 40° C./2M | Complies | Complies | Complies | 4.26 | 94.16 | 3.48 | 79.10 |
| EDTA | 25° C./1M | Complies | Complies | Complies | 4.01 | 97.26 | 2.96 | 87.80 |
| (0.032 | 25° C./2M | Complies | Complies | Complies | 4.17 | 96.46 | 2.85 | 87.00 |
| mg/mL) | | | | | | | | |

| | Analytical Data | | | |
|---|---|---|---|---|
| | | | | Particulate matter |
| | | | | For ≥10 μm: NMT |
| | Related Substances | | | 25 particles per mL |
| | | Each individual | Total impurity | For ≥25 μm: |
| Trial | Impurity F | unspecified impurity | (D + F Imp) | Not More Than |
| details | NMT 7.5% | NMT 0.2% | NMT 10.0% | 3 particles per mL |
| Trial | 2.454 | ND | ND | 10 μm: 02 |
| with | | | | 25 μm: 0 |
| Disodium | 3.931 | 0.016(RRT-0.51) | 0.024 | 10 μm: 02 |
| EDTA | | 0.008(RRT-0.62) | | 25 μm: 00 |
| (0.032 | 5.388 | ND | ND | 10 μm: 05 |
| mg/mL) | | | | 25 μm: 00 |
| | 2.346 | 0.010(RRT-0.51) | 0.010 | 10 μm: 02 |
| | | | | 25 μm: 01 |
| | 2.684 | ND | ND | 10 μm: 01 |
| | | | | 25 μm: 00 |

TABLE 23
Epinephrine 64 mcg/mL with 0.032 mg/ml edetate disodium dihydrate (EDTA) and 0.05 mg/mL of SMBS

| | | Analytical Data | | | | | | |
|---|---|---|---|---|---|---|---|---|
| | | | Color and | | | | | |
| | | | achromicity | | | | | |
| | | | of solution | Clarity of | | | | |
| | | | Color of the | solution | | Assay | | |
| | | Description | solution should | Clear solution | | L- | D- | |
| | | A clear and | not be more | essentially | pH | Epinephrine | Epinephrine | SMBS |
| Trial | | colorless solution | intense than | free from visible | Between | 90.0 to | NMT | 10.0 to |
| details | Conditions | filled in a bag | Purified water. | particles | 3.0 and 5.0 | 110.0% | 5.5% | 110.0% |
| Trial | Initial | Complies | Complies | Complies | 4.06 | 94.31 | 3.01 | 88.9 |
| with | 40° C./1M | Complies | Complies | Complies | 4.04 | 95.90 | 3.50 | 80.10 |
| Disodium | 40° C./2M | Complies | Complies | Complies | 4.20 | 93.82 | 3.53 | 74.60 |
| EDTA | 25° C./1M | Complies | Complies | Complies | 3.99 | 97.24 | 3.16 | 85.50 |
| (0.032 | 25° C./2M | Complies | Complies | Complies | 4.14 | 95.97 | 2.94 | 83.3 |
| mg/mL) | | | | | | | | |

| | Analytical Data | | | |
|---|---|---|---|---|
| | | | | Particulate matter |
| | Related Substances | | | For ≥10 μm: NMT |
| | | | Total impurity | 25 particles per mL |
| | | Each individual | (Excluding Imp F and D- | For ≥25 μm: |
| Trial | Impurity F | unspecified impurity | Epinephrine) | Not More Than |
| details | NMT 7.5% | NMT 0.2% | NMT 10.0% | 3 particles per mL |
| Trial | 2.32 | 0.037 (RRT-1.11) | 0.048 | 10 μm: 05 |
| with | | 0.011 (RRT-0.86) | | 25 μm: 00 |
| Disodium | 3.715 | 0.025 | 0.069 | 10 μm: 08 |
| EDTA | | | | 25 μm: 01 |
| (0.032 | 5.037 | ND | ND | 10 μm: 01 |
| mg/mL) | | | | 25 μm: 00 |
| | 2.351 | 0.030(RRT-1.08) | 0.030 | 10 μm: 10 |
| | | | | 25 μm: 02 |
| | 2.561 | ND | ND | 10 μm: 05 |
| | | | | 25 μm: 00 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed:

1. A pharmaceutical premix formulation comprising:
about 10 micrograms per milliliter (mcg/ml) to about 70 mcg/ml epinephrine
a salt,
edetate disodium dihydrate (EDTA), and
sodium metabisulfite,
wherein the formulation is an aqueous, premix formulation and has a pH of about 2 to about 6.

2. The pharmaceutical premix formulation of claim 1, comprising about 16 mcg/ml to about 64 mcg/ml epinephrine.

3. The pharmaceutical premix formulation of claim 1, comprising about 16 mcg/ml epinephrine.

4. The pharmaceutical premix formulation of claim 1, comprising about 32 mcg/ml epinephrine.

5. The pharmaceutical premix formulation of claim 1, comprising about 64 mcg/ml epinephrine.

6. The pharmaceutical premix formulation of claim 1, wherein the salt is sodium chloride.

7. The pharmaceutical premix formulation of claim 6, comprising about 8.5 mg/ml to about 9.5 mg/ml sodium chloride; about 8.7 mg/ml to about 9.3 mg/ml sodium chloride; or about 9 mg/ml sodium chloride.

8. The pharmaceutical premix formulation of claim 6, comprising about 0.9% sodium chloride by weight per volume.

9. The pharmaceutical premix formulation of claim 1, wherein the pH is about 2.2 to about 5.5.

10. The pharmaceutical premix formulation of claim 1, comprising about 0.01 to about 0.3 mg/ml edetate disodium dihydrate; or about 0.032 mg/ml edetate disodium dihydrate.

11. The pharmaceutical premix formulation of claim 1, wherein the formulation further comprises a molar ratio of epinephrine to sodium metabisulfite, measured as sulfite-equivalents, in the range of 0.17 to 0.66.

12. The pharmaceutical premix formulation of claim 1, comprising about 0.03 to about 0.07 mg/ml sodium metabisulfite; or about 0.05 mg/ml sodium metabisulfite.

13. A pharmaceutical premix formulation consisting essentially of:
epinephrine;
sodium chloride;
edetate disodium dihydrate; and
sodium metabisulfite;
wherein the pharmaceutical premix formulation is an aqueous, premix
formulation and has a pH of about 2.2 to about 5.5, and wherein the formulation comprises a molar ratio of epinephrine to sodium metabisulfite, measured as sulfite-equivalents, in the range of 0.17 to 0.66.

14. The pharmaceutical premix formulation of claim 13, comprising about 10 mcg/ml to about 70 mcg/ml epinephrine.

15. The pharmaceutical premix formulation of claim 13, comprising about 8.4 mg/ml to about 9.4 mg/ml sodium chloride.

16. The pharmaceutical premix formulation of claim 13, comprising about 0.01 to about 0.3 mg/ml edetate disodium dihydrate and, about 0.03 to about 0.07 mg/ml sodium metabisulfite.

17. The pharmaceutical premix formulation of claim 13, which is stable at about 25 degrees Celsius for at least about 6 months or for at least about 12 months.

18. The pharmaceutical premix formulation of claim 13, which is stable for more than about 4 hours at about 21-22 degrees Celsius, and/or is stable for more than about 24 hours at about 4 degrees Celsius.

19. A pharmaceutical premix formulation consisting essentially of:
about 16 mcg/ml epinephrine, about 32 mcg/ml epinephrine, or about 64 mcg/ml epinephrine;
about 0.9% sodium chloride;
about 0.032 mg/ml edetate disodium dihydrate; and
about 0.05 mg/ml sodium metabisulfite;
wherein the pharmaceutical premix formulation is an aqueous, premix formulation and has a pH of about 2.2 to about 5.5.

20. A flexible container comprising the pharmaceutical premix formulation of claim 1.

21. The flexible container of claim 20, which is polypropylene (PP), polyamide (PA), or polyethylene (PE).

22. The flexible container of claim 20, having a volume of about 100 mL or about 250 mL.

23. A system comprising an oxygen scavenger and the pharmaceutical premix formulation of claim 1.

24. The system of claim 23, wherein the oxygen scavenger comprises micronized iron.

25. A method of treating hypotension in a human subject in need thereof, the method comprising intravenously administering the pharmaceutical premix formulation of claim 1 to the human subject in need thereof, wherein the pharmaceutical premix formulation is not diluted prior to intravenous administration to the human subject.

26. The method of claim 25, wherein the human subject has anaphylaxis or hypotension associated with septic shock.

27. The method of claim 25, wherein the formulation is administered as an intravenous bolus, or as a continuous intravenous infusion.
